(19) United States
(12) Patent Application Publication
(10) Pub. No.: US 2024/0382506 A1
(43) Pub. Date: Nov. 21, 2024

(54) Means and Methods to Regulate Gut Dysbiosis

(71) Applicants: VIB VZW, Gent (BE); Universiteit Gent, Gent (BE)

(72) Inventors: Kodimangalam Ravichandran, Gent (BE); Christopher Anderson, Gent (BE)

(21) Appl. No.: 18/570,876

(22) PCT Filed: Jun. 20, 2022
(86) PCT No.: PCT/EP2022/066746

§ 371 Date: Dec. 15, 2023

Related U.S. Application Data
(60) Provisional application No. 63/212,242, filed on Jun. 18, 2021.
(60) Provisional application No. 63/212,245, filed on Jun. 18, 2021.

(51) Int. Cl. A61K 31/704; A61K 31/7036; A61K 45/06; A61P 1/00; A61P 31/04

(52) U.S. Cl. CPC A61K 31/704; A61K 31/7036; A61K 45/06; A61P 1/00; A61P 31/04

(57) ABSTRACT

The present invention relates to the field of microbial growth, more specifically microbial overgrowth, even more specifically overgrowth of Enterobacteriaceae, specifically under conditions of apoptosis in the gut such as IBD, colitis, chemotherapy and food borne bacterial infections. The present invention provides inhibitors of a bacterial target, pyruvate formate lyase, which can be used to regulate the overgrowth of Enterobacteriacea.

### CROSS REFERENCE TO RELATED APPLICATIONS

This application is a national phase entry under 35 U.S.C. § 371 of International Patent Application PCT/EP2022/066746, filed Jun. 20, 2022, designating the United States of America and published in English as International Patent Publication WO 2022/263678 on Dec. 22, 2022, which claims the benefit under Article 8 of the Patent Cooperation Treaty to U.S. Provisional Patent Application Ser. No. 63/212,242, filed Jun. 18, 2021, and U.S. Provisional Patent Application Ser. No. 63/212,245, filed Jun. 18, 2021, the entireties of which are hereby incorporated by reference.

### FIELD OF THE INVENTION

The present invention relates to the field of microbial growth, more specifically microbial dysbiosis or microbial overgrowth, even more specifically overgrowth of Enterobacteriaceae, specifically under conditions of apoptotic cell death in the gut due to disease indications such as IBD, colitis, chemotherapy and food borne bacterial infections. The present invention provides genetic inhibitors of a bacterial target, pyruvate formate lyase, which can be used to regulate the overgrowth of Enterobacteriacea in conditions of IBD, colitis, chemotherapy and food borne bacterial infections.

### Introduction to the Invention

Regulated cell death is an integral part of life, having broad impacts on organism development and homeostasis¹. Malfunctions within the regulated cell death process, including the clearance of dying cells, can manifest in a diverse range of pathologies throughout various tissues such as the gastrointestinal tract². Apoptosis is the primary form of regulated cell death during development and homeostasis³, though additional forms of cell death, including lytic pyroptosis and necroptosis, have substantial roles in various disease contexts^{4,5}. A fascinating, yet loosely defined, relationship exists between gastrointestinal pathologies, mammalian cell death, and enteric bacteria⁶⁻⁸. For example, many foodborne bacterial pathogens, including the non-typhoidal serovars of Salmonella enterica (Salmonella), have direct and indirect mechanisms to induce apoptotic and pyroptotic forms of mammalian cell death⁹. Additionally, patients suffering from inflammatory bowel disease (IBD) show higher intestinal apoptotic cell death during disease flare ups¹⁰, and exhibit ‘dysbiosis’ with an increase in the Enterobacteriaceae (including Escherichia coli)¹¹. Further, cytotoxic cancer chemotherapeutics cause substantial gastrointestinal toxicity and mucositis in patients¹² that increases the risk of developing bacterial infections¹³, which is compounded by chemotherapy-induced neutropenia¹⁴. Although a clear relationship exists between increases in Proteobacteria (which includes the Enterobacteriaceae) and diseases such as IBD and intestinal cancers in human patients¹⁵, a direct link between dying mammalian cells and subsequent bacterial outgrowth remains unexplored. Intriguingly, mammalian cells undergoing regulated cell death release a select set of proteins¹⁶, lipids¹⁷, and nucleotides (such as ATP)¹⁸. Moreover, death-dependent metabolites can act as signals to neighboring cells to promote the clearance of dying corpses and aid in the resolution of inflammation^{19,20}. Given the close relationship between mammalian cell death and bacteria-centric pathologies, we tested whether molecules released from apoptotic mammalian cells (e.g. during foodborne infections, gut inflammation, or cancer therapies) might provide direct fuel for bacterial growth. There is a need to identify bacterial targets, particularly Enterobacteriacea targets, which can be inhibited for regulating gut dysbiosis. The present invention satisfies this need and provides the bacterial pyruvate formate lyase as a new target for regulating gut dysbiosis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1J. Regulated mammalian cell death enhances bacterial growth

FIG. 1A, Schematic of the cell death induction approach in primary colonocytes from distinct mouse strains. FIG. 1B, TUNEL positive cells from C57BL/6 colonic explants quantified using automated slide scanning image analysis from paired samples. Live (DMSO-treated) or Stauro (2 μM) samples were treated for 8 hours ex vivo. *p≤0.05, paired t-test. n=4 colonocyte explants. FIG. 1C, Cleaved caspase 3 staining of DMSO (Live, top) and Stauro (bottom) treated samples. FIG. 1D, Activated caspase-3 units (colorimetric assay) of C57BL/6 colonic explants. Live (DMSO-treated) or Stauro (2 μM) samples were treated for 8 hours ex vivo. *p≤0.05, paired t-test, n=3 independent colonic explants. FIG. 1E, Colony forming units (CFU) of Salmonella after 9 hours of aerobic growth in fresh media or supernatants collected from Live (vehicle-treated) or Stauro-treated C57BL/6 colonic explants. Colonic explants were either treated with the pan-Caspase inhibitor QVD alone, Stauro alone, or QVD and Stauro together (n=5-10 per condition). *p≤0.05, one-way ANOVA with Tukey's multiple comparisons test. FIG. 1F, Salmonella CFU following 9 hours of aerobic growth in supernatants of Vil-Cre^{+/-} Caspase 3/7^{fl/fl} colonic explants (as in FIG. 1E). Colonic explants were treated with or without Stauro before supernatant collection (n=7-8 per condition). ns p>0.05, unpaired Student's t-test. FIG. 1G, Activated caspase-3 units (colorimetric assay) of C57BL/6 colonic explants. Live (water-treated) or Doxo (20 μg/ml) samples were treated for 6 hours ex vivo *p≤0.05, paired t-test, n=3 independent colonic explants. FIG. 1H, CFU of Salmonella after 9 hours of aerobic growth in fresh media+20 μg/ml Doxo or C57BL/6 colonic explant supernatant following 20 μg/ml Doxo treatment ex vivo. n=3. Median is shown. *p≤0.05, unpaired Student's t-test. FIG. 1I, CFU of Salmonella and E. coli (strain HS) after 9 hours of aerobic growth in media with or without Doxo. ***p≤0.0005, two-way ANOVA with Tukey's multiple comparisons test. FIG. 1J, Salmonella CFU following 9 hours aerobic growth in supernatants of Vil-Cre^{+/-} Caspase 3/7^{fl/fl} or littermate control colonic explants (left); media, RIPK1 kinase dead (RIPK1^{KD}), MLKL^{-/-}, or control colonic explants (center); GasderminD^{-/-}, Caspase 1/11^{-/-}, or control colonic explants (right). Colonic explants were treated with or without Doxo for 6 hours ex vivo before supernatant collection (n=5-14 independent explants). ns p>0.05, **p≤0.005, ***p≤0.0005, one-way ANOVA with Tukey's multiple comparisons test (center, right) or unpaired Student's t-test (left).

FIGS. 2A-2E. Death triggers induce bacterial growth

FIG. 2A, Cartoon schematic of in vitro cell line approach. FIG. 2B, CT26 cell death, as measured by flow cytometry. CT26:FADD cell death with or without caspase inhibition via QVD (left, n=5 per condition) 5 hours post-death induction. CT26 cell death (center, n=4) 24 hours post-600 mJ cm⁻² UV treatment. CT26 cell death (right, n=3) 24 hours post-Staurosporine (Stauro) treatment. Mean±standard error of the mean (s.e.m) are shown. ***p≤0.0005, one-way ANOVA with Tukey's multiple comparisons test (left), Student's t-test (center, right). FIG. 2C, Salmonella colony forming units (CFU) (left, n=9-10 per condition) following 8 hours of aerobic growth. **p≤0.005, ***p≤0.0005, one-way ANOVA with Tukey's multiple comparisons test. FIG. 2D, Bacterial CFU of the Salmonella, E. coli (strain HS), or Klebsiella (center, right, n=4 per condition) following 9 hours of aerobic growth. *p≤0.05, **p≤0.005, ***p≤0.0005, multiple Student's t-tests. FIG. 2E, Bacterial CFU of human-derived E. coli isolates (right, n=4 per condition) following 9 hours of aerobic growth. *p≤0.05, **p≤0.005, ***p≤0.0005, multiple Student's t-tests.

FIGS. 3A-3J. Mammalian cell death induces time-dependent bacterial outgrowth

FIG. 3A, CT26: FADD cells were treated overnight with doxycycline to induce construct expression and were then treated with B/B dimerizer, with or without pan caspase inhibitor (QVD), and supernatants were collected. Periodic Salmonella aerobic growth measurements were taken via OD₆₀₀. n=5 per condition. Mean+s.e.m are shown. ***p≤0.0005, two-way ANOVA with Tukey's multiple comparisons test. FIG. 3B, Salmonella aerobic growth in CT26 supernatants with or without 600 mJ cm⁻² UV irradiation using RPMI cell media (left), or media with DMSO vehicle, Stauro, QVD, or Stauro+QVD (right). Bacterial growth was assessed via CFU or repeated OD₆₀₀ measurements as indicated. Mean+s.e.m are shown. * p≤0.05, **p≤0.005, two-way ANOVA with Tukey's multiple comparisons test. FIG. 3C, CT26 cell death (left), as determined by flow cytometry, following 24-hour treatment with 50 μM Pac-1 (apoptosis inducer) and Salmonella aerobic growth (right) in those supernatants as measured by OD₆₀₀ values. Mean+s.e.m are shown. *p≤0.05, two-way ANOVA with Tukey's multiple comparisons test. FIG. 3D, Bacterial aerobic growth in CT26 cell supernatants 24 hours after 600 mJ cm⁻² UV irradiation. Salmonella (left), E. coli strain HS (middle), and Klebsiella (right) growth as measured via OD₆₀₀. Mean+s.e.m are shown. *p≤0.05, **p≤0.005, ***p≤0.0005, two-way ANOVA with Tukey's multiple comparisons test. FIG. 3E, Bacterial aerobic growth in CT26 cell supernatants 24 hours after 1 μM Stauro treatment. Salmonella (left), E. coli strain HS (middle), and Klebsiella (right) growth was measured via OD₆₀₀. Mean+s.e.m are shown. *p≤0.05, **p≤0.005, ***p≤0.0005, two-way ANOVA with Tukey's multiple comparisons test. FIG. 3F, CT26 cell death (left) and Salmonella aerobic growth in CT26 cell supernatants 24 hours after 1 μM Stauro treatment measured via OD₆₀₀ (right). CT26 cells were pretreated for 1 hour and maintained with 30 μM QVD as indicated. Mean+s.e.m are shown. **p≤0.005, ***p≤0.0005, two-way ANOVA with Tukey's multiple comparisons test. FIG. 3G, Total cell death (left) of HCT116 cells 24 hours after 600 mJ cm⁻² UV irradiation. Corresponding Salmonella aerobic growth (right) in HCT116 supernatants as measured by OD₆₀₀. n=3 per condition. Mean+s.e.m are shown. ***p<0.0005, Student's t test (cell death) or two-way ANOVA with Tukey's multiple comparisons test (bacterial growth curve). FIG. 3H, Total cell death (left) of HCT116 cells 24 hours after 1 μM Stauro treatment. Corresponding Salmonella aerobic growth (right) in HCT116 supernatants as measured by OD₆₀₀. n=3 per condition. Mean+s.e.m are shown. **p≤0.005, ***p≤0.0005, Student's t test (cell death) or two-way ANOVA with Tukey's multiple comparisons test (bacterial growth curve). FIG. 3I, Jurkat cell death characterization, as measured by flow cytometry, 4 hours post 150 mJ cm⁻² UV irradiation. Quantification of membrane integrity (left), n=3, Mean+s.e.m are shown. Corresponding Salmonella aerobic CFU (right, n=6-7). *p≤0.05, unpaired Student's t-test. FIG. 3J, CT26 cell death following 3 cycles of freeze-thaw, as measured by flow cytometry. Quantification (left), and the corresponding Salmonella aerobic growth in the supernatants of freeze-thaw conditions (OD₆₀₀) are shown (right, n=4).

FIGS. 4A-4G. Mammalian death-driven bacterial growth is protein-independent

FIG. 4A, Schematic of supernatant manipulations following induction of apoptosis that ruled out proteins as responsible for enhanced bacterial growth. FIG. 4B, Total protein levels in media or CT26 supernatants, with or without FBS, at 24 hours post-Stauro treatment following indicated Proteinase K or filtration strategies as determined by BCA total protein assay. n=4 per condition. Mean+s.e.m are shown. FIG. 4C, CFU of Salmonella in media controls or CT26 supernatants, without FBS, following Stauro treatment. Media and CT26 supernatants were sequentially filtered using the indicated filter sizes. n=3 per filter size, 9 hours of aerobic growth. Median is shown. **p≤0.005, ***p≤0.0005, multiple Student's t-tests. FIG. 4D, CFU of Salmonella in media controls or CT26 supernatants, without FBS, following Stauro treatment. Following collection, media and CT26 supernatants were sequentially filtered down to <10 kD, treated or untreated with Proteinase K, and Proteinase K was removed with a 3 kD filter. n=6-8 per condition, 9 hours of aerobic growth. ***p≤0.0005, one-way ANOVA with multiple comparisons test. FIG. 4E, CFU of Salmonella in media controls or indicated CT26 supernatants, without FBS, following Stauro treatment. Following collection, media and CT26 supernatants were boiled or left at room temperature. n=5 per condition, 9 hours of aerobic growth. ***p≤0.0005, one-way ANOVA with Tukey's multiple comparisons test. FIG. 4F, CFU of Salmonella after 9 hours of aerobic growth in media or indicated CT26 cell supernatants, containing 10% FBS, treated with Stauro. Media and supernatants were sequentially filtered to <3 kD (as in panel c). n=5 per condition. *p≤0.05, unpaired Student's t-test. FIG. 4G, CFU of Salmonella after 9 hours of aerobic growth in media or the indicated CT26 cell supernatants cultured without FBS in phenol red-free media. ***p≤0.0005, unpaired Student's t-test.

FIGS. 5A-5N. Mammalian cell death nutrients promote pflB expression and growth in Salmonella

FIG. 5A, Schematic for steps taken to performing Salmonella RNAseq analysis. FIG. 5B, Venn diagram shows differentially regulated Salmonella genes in the two different RNAseq experiments and the list of eight regulated genes shared between the two datasets. FIG. 5C, CFU of WT or ΔcadBA mutant Salmonella (CJA042) following 9 hours of aerobic growth in media or CT26 supernatants collected after Stauro treatment. n=4 per condition. ns p>0.05, two-way ANOVA with Tukey's multiple comparisons test. FIG. 5D, Salmonella cadB expression, as measured by qPCR, in the indicated mammalian cell line supernatant system following the indicated cell death trigger. n=3-4 per condition. Mean+s.e.m are shown. *p≤0.05, **p≤0.005, ***p≤0.0005, one-way ANOVA with Tukey's multiple comparisons test. FIG. 5E, Salmonella pflB expression, as measured by qPCR, in the indicated mammalian cell line supernatant system following the indicated cell death trigger. n=3-8 per condition. Mean+s.e.m are shown. *p≤0.05, **p≤0.005, ***p≤0.0005, one-way ANOVA with Tukey's multiple comparisons test. FIG. 5F, CFU of WT or ΔpflB mutant Salmonella (CJA071) after 9 hours of aerobic growth in media or CT26 supernatants following Stauro treatment. n=4 per condition. ns p>0.05, *p≤0.05, two-way ANOVA with Tukey's multiple comparisons test. FIG. 5G, CFU of WT or ΔpflB mutant Salmonella (CJA071) after 9 hours of anaerobic growth in media or CT26 supernatants following Stauro treatment. n=4 per condition. ns p>0.05, ***p≤0.0005, two-way ANOVA with Tukey's multiple comparisons test. FIG. 5H, Pyruvate concentrations of media +/−10% FBS, live (QVD-treated), apoptotic (following staurosporine or UV irradiation), or necrotic (freeze-thaw) supernatants. n=4-13 per condition. ***p≤0.0005, one-way ANOVA with Tukey's multiple comparisons test. FIG. 5I, Formate concentrations in supernatants derived from live (QVD treated), Stauro treated, UV irradiated, or freeze-thaw CT26 cells. n=4-7 per condition. ns p>0.05, one-way ANOVA with Tukey's multiple comparisons test. FIG. 5J, Pyruvate concentrations of live (QVD-treated), UV-irradiated, or UV-irradiated+Shikonin supernatants. n=4 per condition. Data were normalized to live supernatants. *p≤0.05, one-way ANOVA with Dunnett's multiple comparisons test. FIG. 5K, CT26 cell death, as measured by flow cytometry. CT26 cell death 24 hours post UV irradiation or Staurosporine (Stauro) treatment with or without Skinonin. Mean±s.e.m are shown. n=3-4 per condition. FIG. 5L, CFU of WT Salmonella after 9 hours of anaerobic growth in CT26 supernatants following UV irradiation with or without Shikonin treatment. n=7-9 per condition. **p≤0.005, unpaired t test. FIG. 5M, CFU of WT Salmonella after 9 hours of anaerobic growth in CT26 supernatants following Stauro treatment with or without Skinonin treatment and pyruvate supplementation. n=8-12 per condition. ns p>0.05, *p≤0.05, ***p≤0.0005, one-way ANOVA with Tukey's multiple comparisons test. FIG. 5N, CFU of WT or ΔpflB mutant Salmonella (CJA071) after 9 hours of anaerobic growth in CT26 supernatants following Stauro treatment with Skinonin treatment with or without pyruvate supplementation. n=5-6 per condition. ns p>0.05, *p≤0.05, two-way ANOVA with Sidak's multiple comparisons test.

FIGS. 6A-6F. Pannexin-1 dependent metabolites enhance Enterobacteriaceae growth

FIG. 6A, Percent apoptosis (via Annexin V staining) and activation of Panx1 channel (via ToPo3 dye uptake) in control or Panx1-DN expressing Jurkat cells following UV irradiation (left). n=4, ns p>0.05, ***p≤0.0005 two-way ANOVA with Sidak's multiple comparisons test. WT or ΔpflB (CJA071) Salmonella CFU after 9 hours of anaerobic growth in fresh media controls or the indicated apoptotic Jurkat supernatants (right). n=11-12 per condition, ns p>0.05, ***p≤0.0005 two-way ANOVA with Sidak's multiple comparisons test. FIG. 6B, Salmonella CFU after 9 hours of anaerobic growth in media supplemented with the indicated dilutions of ‘MeMix-6’ metabolites. n=4-14 per condition. ***p≤0.0005 one-way ANOVA with Dunnett's multiple comparisons test compared to media control. ‘MeMix-6’ formulation used: Spermidine (3.0 nM); FBP: fructose 1,6-bisphosphate (5 nM); DHAP: dihydroxyacetone phosphate (0.36 μM); UDG-glucose (20 nM); GMP: guanosine monophosphate (21 nM); IMP: inosine monophosphate (33 nM). 1× concentrations were based on previously identified concentrations of indicated metabolites in apoptotic supernatants. Right, WT or pflB mutant (CJA071) CFU following 9 hours of anaerobic growth. ns p>0.05, ***p≤0.0005 two-way ANOVA with Sidak's multiple comparisons test. FIG. 6C, Salmonella pflB or cadB gene expression in the absence or presence of the MeMix, as measured by qPCR. n=4 per condition. ns p>0.05, two-way ANOVA with Sidak's multiple comparisons test. FIG. 6D, CFU after 9 hours of anaerobic growth in media supplemented with UDPg and FBP of WT with empty vector (WT), pflB mutant with empty vector (ΔpflB), or complemented pflB mutant (pflB+) following 9 hours of anaerobic growth with or without 0.2% arabinose. ns p>0.05, *p≤0.05, **p≤0.005, ***p≤0.0005 two-way ANOVA with Tukey's multiple comparisons test. FIG. 6E, CFU of the indicated strain of E. coli (LF82, CCR20, UTI189, HS) after 7 hours of anaerobic growth in media supplemented with or without UDPg & FBP. *p≤0.05, ***p≤0.0005 two-way ANOVA with Sidak's multiple comparisons test. FIG. 6F, Fructose 1,6-bisphosphate concentrations in fresh media or supernatants derived from live (QVD treated), or Stauro treated CT26 cells. n=4 per condition. ***p≤0.0005, one-way ANOVA with Tukey's multiple comparisons test.

FIGS. 7A-7N. PflB promotes Salmonella fitness during foodborne infection

FIG. 7A, Schematic of a foodborne Salmonella infection model for competitive infections. Germ-free mice did not receive oral streptomycin treatment and were infected with 1e6 CFU/mouse. FIG. 7B, Salmonella burden of WT (black) or ΔcadBA (CJA033, green) in the indicated tissue of C57BL/6 conventional specific pathogen free (SPF) mice at day 4 post-infection. WT and mutant Salmonella connected with dotted lines originate from the same mouse. The median competitive index (WT/ΔcadBA) is listed below each tissue. n=7 female mice from 2 cohorts. Tissue samples were excluded if either total or mutant strain burden fell below the limit of detection. ns p>0.05, *p≤0.05, Wilcoxon Signed Rank Test with theoretical median of 1 using the calculated competitive index from each mouse. FIG. 7C, Pyruvate concentrations in fresh luminal samples from uninfected (streptomycin treated) or WT Salmonella infected C57BL/6 conventional SPF mice at day 4 post-infection. n=4-7 mice per condition from 3 cohorts. *p≤0.05, Mann Whitney test. FIG. 7D, Salmonella burden of WT (black) or ΔpflB (CJA057, blue) in the indicated tissue of C57BL/6 conventional SPF mice at day 4 post-infection. WT and mutant Salmonella connected with dotted lines originate from the same mouse. The median competitive index (WT/ΔpflB) is listed below each tissue. n=8 female mice from 2 cohorts. *p≤0.05, Wilcoxon Signed Rank Test with theoretical median of 1 using the calculated competitive index from each mouse. FIG. 7E, Salmonella burden of WT (black) or ΔpflB (CJA057, blue) in C57BL/6 conventional SPF mice at day 2 post-infection. The median competitive index (WT/ΔpflB) is listed below each tissue. n=6 female mice from 2 cohorts. *p≤0.05, Competitive index was calculated using the Wilcoxon Signed Rank Test with theoretical median of 1 for each mouse. FIG. 7F, Salmonella burden of WT (black) or ΔpflB (CJA057, blue) in the indicated tissue of C57BL/6 Germ-Free mice at day 2 post-infection. The median competitive index (WT/ΔpflB) is listed below each tissue. n=9 female mice from 2 cohorts. Tissue samples were excluded if either total or mutant strain burden fell below the limit of detection. *p≤0.05, **p≤0.005, Wilcoxon Signed Rank Test with theoretical median of 1 using the calculated competitive index from each mouse. FIG. 7G, Salmonella burden of WT (black), ΔSPI-1ΔSPI-2 (CJA077, orange), or ΔpflB (CJA057, blue) in the indicated tissue of C57BL/6 SPF mice at day 4 post-infection. n=7-15 per strain from 4 cohorts. ns p>0.05, **p≤0.005, Kruskal-Wallis with Dunn's multiple comparisons test. FIG. 7H, Competitive index of either WT Salmonella compared to ΔpflB (CJA057) (black) or ΔSPI-1ΔSPI-2 (CJA077) compared to ΔSPI-1ΔSPI-2ΔpflB (CJA081) (orange) at day 4 post-infection. n=14 female mice from 4 cohorts. ns p>0.05, *p≤0.05, Mann Whitney test. FIG. 7I, Salmonella burden of WT (black) or ΔpflB (CJA057, blue) in the indicated tissue of Caspase 1/11^{-/-} conventional SPF mice at day 4 post-infection. n=8 male and female mice from 2 cohorts. FIG. 7J, Salmonella burden of WT (black) or ΔpflB (CJA057, blue) in Caspase 1/11 KO SPF mice. WT and mutant Salmonella connected with dotted lines come from the same mouse. The median competitive index is listed below. n=7 male and female mice from 2 cohorts. *p≤0.05, Wilcoxon Signed Rank Test with theoretical median of 1 using the calculated competitive index from each mouse. FIG. 7K, Salmonella burden in the indicated tissue of Caspase 3/7^{fl/fl} control or Vil-Cre^{+/-} Caspase 3/7^{fl/fl} mice at day 4 post-infection. n=11 female Caspase 3/7^{fl/fl} control mice, n=7 female Vil-Cre^{+/-} Caspase 3/7^{fl/fl} mice from 3 cohorts. FIG. 7L, Competitive index of WT Salmonella compared to ΔpflB (CJA057) in the indicated tissue of Caspase 3/7^{fl/fl} control or Vil-Cre^{+/-} Caspase 3/7^{fl/fl} mice at day 4 post-infection. n=8 female Caspase 3/7^{fl/fl} control mice, n=5 female Vil-Cre^{+/-} Caspase 3/7^{fl/fl} mice from 2 cohorts. ns p>0.05, *p≤0.05, Mann Whitney test with each tissue analyzed separately. FIG. 7M, Salmonella burden of WT (black) or ΔpflB (CJA057, blue) in the indicated tissue of Vil-Cre^{+/-} Caspase 3/7^{fl/fl} conventional SPF mice at day 4 post-infection. n=5 female mice from 2 cohorts. FIG. 7N, Competitive index of WT Salmonella compared to ΔpflB (CJA057) in the ilea of Caspase 3/7^{fl/fl} control or Vil-Cre^{+/-} Caspase 3/7^{fl/fl} mice at day 4 post-infection. n=8 female Caspase 3/7^{fl/fl} control mice, n=5 female Vil-Cre^{+/-} Caspase 3/7^{fl/fl} mice from 2 cohorts. *p<0.05, Mann Whitney test.

FIGS. 8A-8K. TNF & A20-dependent cell death enhances Enterobacteriaceae growth

FIG. 8A, Schematic of the in vitro approach for using A20 knockout HCT116 cells. FIG. 8B, Total cell death of control or A20 KO HCT116 cells 24 hours after 100 ng/ml human TNF stimulation, and the inhibition of apoptosis by QVD treatment. n=4 per condition. Mean+s.e.m are shown. ns p>0.05, ***p≤0.0005, one-way ANOVA with Sidak's multiple comparisons test. FIG. 8C, Salmonella CFU following 9 hours of aerobic growth in Ctrl or A20 KO HCT116 cell supernatants with or without 100 ng/ml human TNF treatment. n=9-13 per condition. ns p>0.05, ***p≤0.0005, one-way ANOVA with Tukey's multiple comparisons test. FIG. 8D, Salmonella pflB gene expression following mid-log aerobic growth in the indicated HCT116 cell supernatants. pflB expression was normalized to 1 in control HCT116 supernatants with and without 100 ng/ml human TNF. n=7 per condition. Mean+s.e.m are shown. ns p>0.05, ***p≤0.0005, one-way ANOVA with Sidak's multiple comparisons test. FIG. 8E, CFU of E. coli (strain HS) and Klebsiella measured after 9 hours of aerobic growth in supernatants collected from Ctrl or A20 KO HCT116 cells with 100 ng/ml human TNF treatment. n=7 per condition per strain. *p≤0.05, ***p≤0.0005 unpaired Student's t test. FIG. 8F, Salmonella cadB gene expression, as measured by qPCR, following mid-log growth in the indicated HCT116 cell supernatants. cadB expression was normalized to 1 in Ctrl HCT116 supernatants with and without 100 ng/ml human TNF. n=7 per condition. Mean+s.e.m are shown. ns p>0.05, ***p≤0.0005, one-way ANOVA with Sidak's multiple comparisons test. FIG. 8G, Schematic of in vivo infections in control mice versus mice lacking A20 in intestinal epithelial cells. FIG. 8H, A20^{fl/fl} control or Vil-Cre^{+/-} A20^{fl/fl} mice were infected (as above). Daily weights were recorded and % body weight was calculated based on weight at day 0 for each mouse. n=12 male and female A20^{fl/fl} control mice. n=6 male and female Vil-Cre^{+/-} A20^{fl/fl} mice from 2 cohorts. Mean+s.e.m are shown. ***p≤0.0005, two-way ANOVA with Sidak's multiple comparisons test. FIG. 8I, Activated caspase-3 units in the ileum at day 2 or 3 post-infection using A20^{fl/fl} control or Vil-Cre^{+/-} A20^{fl/fl} mice. Day 2: n=4 male A20^{fl/fl} control mice, n=6 male Vil-Cre^{+/-} A20^{fl/fl} mice from 2 cohorts. Day 3: n=4 female A20^{fl/fl} control mice, n=4 female Vil-Cre^{+/-} A20^{fl/fl} mice from 2 cohorts. ns p>0.05, *p≤0.05, two-way ANOVA with Sidak's multiple comparisons test. FIG. 8J, Salmonella burden in the ilea of A20^{fl/fl} control or Vil-Cre^{+/-} A20^{fl/fl} mice at day 2 or 3 post-infection. Day 2: n=6 male and female A20^{fl/fl} control mice, n=7 male and female Vil-Cre^{+/-} A20^{fl/fl} mice from 2 cohorts. Day 3: n=9 male and female A20^{fl/fl} control mice, n=4 male and female Vil-Cre^{+/-} A20^{fl/fl} mice from 2 cohorts. ns p>0.05, **p≤0.005, Mann Whitney test. FIG. 8K, Competitive index of WT Salmonella vs ΔpflB (CJA057) in the ilea of A20^{fl/fl} control or Vil-Cre^{+/-} A20^{fl/fl} mice. Day 2: n=6 male and female A20^{fl/fl} control mice, n=7 male and female Vil-Cre^{+/-} A20^{fl/fl} mice from 2 cohorts. Day 3: n=7 male and female A20^{fl/fl} control mice, n=4 male and female Vil-Cre^{+/-} A20^{fl/fl} mice from 2 cohorts. ns p>0.05, *p≤0.05, Mann Whitney test.

FIGS. 9A-9N. Intestinal epithelial cell apoptosis fuels Salmonella growth in vivo

FIG. 9A, Schematic of the in vivo model of Doxo treatment followed by Salmonella infection. FIG. 9B, Schematic of in vivo Doxorubicin (Doxo) treatment followed by E. coli infection. FIG. 9C, Left: Colon length (cm) of vehicle control (water)-treated mice or mice given Doxo and a representative image of colon shortening. n=7 female C57BL/6 and Caspase 3/7^{fl/fl} control mice, n=12 female C57BL/6 and Caspase 3/7^{fl/fl} mice+Doxo, n=6 female Vil-Cre^{+/-} Caspase 3/7^{fl/fl} mice+Doxo from 3 cohorts. **p≤0.005, one-way ANOVA with Tukey's multiple comparisons test. Right: Cecal weight (in grams) from vehicle-treated (water) control mice or mice given Doxo. n=15 female C57BL/6 and Caspase 3/7^{fl/fl} control mice, n=25 female C57BL/6 and control Caspase 3/7^{fl/fl} mice+Doxo, n=8 female Vil-Cre^{+/-} Caspase 3/7^{fl/fl} mice+Doxo from 3 cohorts. **p≤0.005, ***p≤0.0005, one-way ANOVA with Tukey's multiple comparisons test. FIG. 9D, Salmonella burden in the indicated tissues of C57BL/6 mice either vehicle (water) treated control or Doxo-treated, on day 1 post-infection. n=8 female mice per condition from 2 cohorts. ***p≤0.0005, Mann-Whitney test with each tissue analyzed separately. FIG. 9E, Salmonella burden in the indicated tissues of either Caspase 3/7^{fl/fl} control or Vil-Cre^{+/-} Caspase 3/7^{fl/fl} mice treated with Doxo, on day 1 post-infection. n=16 female control Caspase 3/7^{fl/fl}, n=9 female Vil-Cre^{+/-} Caspase 3/7^{fl/fl} mice from 3 cohorts. *p≤0.05, Mann-Whitney test with each tissue analyzed separately. FIG. 9F, Cecal weight of uninfected C57BL/6 mice with or without Doxo treatment, 2 days post-treatment. n=4 female C57BL/6, n=7 female C57BL/6 mice+Doxo from 2 cohorts. *p≤0.05, unpaired Student's t test. FIG. 9G, Colon length of uninfected C57BL/6 mice with or without Doxo treatment, 2 days post-treatment. n=4 female C57BL/6, n=7 female C57BL/6 mice+Doxo from 2 cohorts. ***p≤0.0005, unpaired Student's t test. FIG. 9H, Activated caspase-3 units in the ilea of C57BL/6 mice with or without Doxo treatment of uninfected mice normalized per gram of tissue, 2 days post-treatment. n=4 female C57BL/6, n=7 female C57BL/6 mice+Doxo from 2 cohorts. **p≤0.005, unpaired Student's t test. FIG. 9I, CFU of endogenous Enterobacteriaceae CFU in the indicated tissues of uninfected C57BL/6 mice with or without Doxo treatment, 2 days post-treatment. n=4 female C57BL/6, n=7 female C57BL/6 mice+Doxo from 2 cohorts. *p≤0.05, Mann-Whitney test with each tissue analyzed separately. FIG. 9J, Competitive index of WT Salmonella vs ΔpflB (CJA057) in the indicated tissues of control or Vil-Cre^{+/-} Caspase 3/7^{fl/fl} mice at day 1 post-infection. n=14 female C57BL/6 and Caspase 3/7^{fl/fl} ‘ctrl’ mice, n=23 female C57BL/6 and Caspase 3/7^{fl/fl} ‘ctrl+Doxo’, n=9 female Vil-Cre^{+/-} Caspase 3/7^{fl/fl} mice+Doxo from 3 cohorts. Tissue samples were excluded if either total or mutant strain burden fell below the limit of detection. ns p>0.05, *p≤0.05, **p≤0.005, ***p≤0.0005, Kruskal-Wallis test with each tissue analyzed separately. FIG. 9K, Salmonella burden in the indicated tissues of either control or Vil-Cre^{+/-} Caspase 3/7^{fl/fl} mice given vehicle, on day 1 post-infection. n=4 Caspase 3/7^{fl/fl} control mice, n=4 female Vil-Cre^{+/-} Caspase 3/7^{fl/fl} mice from 2 cohorts. ns p>0.05, Mann-Whitney test with each tissue analyzed separately. FIG. 9L, Cecal weight (g) from vehicle-treated (water) control mice or mice given Doxo. n=12 female control Panx1^{+/+}, n=18 female control Panx1^{+/+}+Doxo, n=11 female Panx1^{-/-}+Doxo mice from 6 cohorts. FIG. 9M, Salmonella burden in the indicated tissues of either Panx1^{+/+} control or Panx1^{-/-} mice treated with Doxo, on day 1 post-infection. n=8 female control Panx1^{+/+}, n=11 female Panx1^{-/-} mice from 6 cohorts. ns p>0.05, ***p≤0.0005, Mann-Whitney test with each tissue analyzed separately. FIG. 9N, Competitive index of WT Salmonella vs ΔpflB (CJA057) in the indicated tissues of either Panx1^{+/+} control or Panx1^{-/-} mice treated with Doxo, on day 1 post-infection. n=7 female control Panx1^{+/+}, n=6 female Panx1^{-/-} mice from 4 cohorts. ns p>0.05, ***p≤0.0005, Mann-Whitney test with each tissue analyzed separately.

### DETAILED DESCRIPTION OF THE INVENTION

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

To facilitate an understanding of the present technology, a number of terms and phrases are defined below. Additional definitions are set forth throughout the detailed description.

### Definitions

As used herein, the terms “subject” and “patient” refer to any animal, such as a mammal like a dog, cat, bird, pig, horse, cow, livestock, and preferably a human.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (e.g., minimize or lessen) the targeted pathologic condition or disorder. Those in need of treatment include those already with the disorder as well as those prone to have the disorder or those in whom the disorder is to be prevented.

A molecule that “specifically binds to” or is “specific for” another molecule is one that binds to that particular molecule without substantially binding to any other molecule.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the term “administration” refers to the act of giving a drug, prodrug, antisense oligonucleotide, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “protein,” “polypeptide,” and “peptide” refer to a molecule comprising amino acids joined via peptide bonds. In general, “peptide” is used to refer to a sequence of 20 or less amino acids and “polypeptide” is used to refer to a sequence of greater than 20 amino acids.

As used herein, the term, “synthetic polypeptide,” “synthetic peptide”, and “synthetic protein” refer to peptides, polypeptides, and proteins that are produced by a recombinant process (i.e., expression of exogenous nucleic acid encoding the peptide, polypeptide, or protein in an organism, host cell, or cell-free system) or by chemical synthesis.

As used herein, the term “native” (or wild type) when used in reference to a protein refers to proteins encoded by the genome of a cell, tissue, or organism, other than one manipulated to produce synthetic proteins.

As used herein, “domain” (typically a sequence of three or more, generally 5 or 7 or more amino acids) refers to a portion of a molecule, such as proteins or the encoding nucleic acids, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as proteolytic activity. As such, a domain refers to a folded protein structure that retains its tertiary structure independently of the rest of the protein. Generally, domains are responsible for discrete functional properties of proteins, and in many cases may be added, removed or transferred to other proteins without loss of function of the remainder of the protein and/or of the domain.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism). The term “host cell” refers to any cell capable of replicating and/or transcribing and/or translating a heterologous gene. Thus, a “host cell” refers to any eukaryotic or prokaryotic cell, whether located in vitro or in vivo. For example, host cells may be located in a transgenic animal.

As used herein, the term “cell culture” refers to any in vitro culture of cells. Included within this term are continuous cell lines (e.g., with an immortal phenotype), primary cell cultures, finite cell lines (e.g. non-transformed cells), and any other cell population maintained in vitro, including oocytes and embryos.

The term “isolated” when used in relation to a nucleic acid or polypeptide or protein refers to a nucleic acid or polypeptide or protein sequence that is identified and separated from at least one contaminant nucleic acid or polypeptide or protein with which it is ordinarily associated in its natural source. Isolated nucleic acids or polypeptides or proteins are molecules present in a form or setting that is different from that in which they are found in nature. In contrast, non-isolated nucleic acids or polypeptides or proteins are found in the state in which they exist in nature.

The terms “protein” and “polypeptide” refer to compounds comprising amino acids joined via peptide bonds and are used interchangeably. A “protein” or “polypeptide” encoded by a gene is not limited to the amino acid sequence encoded by the gene but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The discussion of the background to the invention herein is included to explain the context of the invention. This is not to be taken as an admission that any of the material referred to was published, known, or part of the common general knowledge in any country as of the priority date of any of the claims. Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

Regulated mammalian cell death has a profound impact on tissue homeostasis and many pathological conditions. A long appreciated, yet elusively defined relationship exists between cell death and gastrointestinal pathologies with an underlying microbial component, but the direct impact of dying mammalian cells on bacterial growth is unclear. Here, using a combination of in vitro and in vivo approaches, we advance a concept that several Enterobacteriaceae, including patient-derived clinical isolates, have an efficient growth strategy to capitalize on soluble factors released from dying gut epithelial cells. Mammalian nutrients released after caspase-3/7-dependent apoptosis boosts the growth of three Enterobacteriaceae family members. The bacterial growth-promoting activity in apoptotic supernatants is observed using primary mouse colonic tissue, murine and human cell lines, multiple apoptotic triggers, and in conventional as well as germ-free mice in vivo. The mammalian cell death nutrients induce a core transcriptional response in pathogenic Salmonella, and we identify the pyruvate formate-lyase encoding pflB gene as a key driver of bacterial colonization. The functional in vivo relevance of gut epithelial cell apoptosis to bacterial growth was revealed via caspase-3/7 deficient mice and pflB mutant bacteria in three contexts: a foodborne infection model, a TNF and A20-dependent cell death model, and a chemotherapy-induced mucositis model. These findings introduce a new layer to the complex host-pathogen interaction, where death-induced nutrient release (which we term DINNR) acts as a source of fuel for intestinal bacteria, with implications for inflammatory diseases of the gut and cytotoxic chemotherapy treatment.

The present application is the first to show specific induced expression of pyruvate formate lyase in bacterial species of the Enterobacteriacea under conditions of apoptosis of gut cells and that inhibition of pyruvate formate lyase can be used to inhibit the growth of Enterobacteriacea in the gut. Accordingly, it is an object of the invention to provide inhibitors of functional expression of the PYRUVATE FORMATE LYASE gene. Such inhibitors can act at the DNA level, or at the RNA (i.e. gene product) level.

With “functional expression” of PYRUVATE FORMATE LYASE, it is meant the transcription and/or translation of a functional gene product. For protein coding genes like PYRUVATE FORMATE LYASE, “functional expression” can be deregulated on at least two levels. First, at the DNA level, e.g. by absence or disruption of the gene, or lack of transcription taking place (in both instances preventing synthesis of the relevant gene product). The lack of transcription can e.g. be caused by loss of function mutations. A “loss-of-function” or “LOF” mutation as used herein is a mutation that prevents, reduces or abolishes the function of a gene product as opposed to a gain-of-function mutation that confers enhanced or new activity on a protein. LOF can be caused by a wide range of mutation types, including, but not limited to, a deletion of the entire gene or part of the gene, splice site mutations, frame-shift mutations caused by small insertions and deletions, nonsense mutations, missense mutations replacing an essential amino acid and mutations preventing correct cellular localization of the product. Also included within this definition are mutations in promoters or regulatory regions of the PYRUVATE FORMATE LYASE gene if these interfere with gene function. A null mutation is an LOF mutation that completely abolishes the function of the gene product. A null mutation in one allele will typically reduce expression levels by 50%, but may have severe effects on the function of the gene product. Note that functional expression can also be deregulated because of a gain of function mutation: by conferring a new activity on the protein, the normal function of the protein is deregulated, and less functionally active protein is expressed. Vice versa, functional expression can be increased e.g. through gene duplication or by lack of DNA methylation.

Second, at the RNA level, e.g. by lack of efficient translation taking place—e.g. because of destabilization of the mRNA (e.g. by UTR variants) so that it is degraded before translation occurs from the transcript. Or by lack of efficient transcription, e.g. because a mutation introduces a new splicing variant.

The enzyme pyruvate formate lyase (abbreviated herein further as pflB) belongs to the enzyme class EC2.3.1.54. Alternative names for this enzyme are formate C-acetyltransferase, formate acetyltransferase, pyruvic formate-lyase. The enzyme belongs to the family of transferases, specifically those acyltransferases transferring groups other than aminoacyl groups. The systematic name of this enzyme class is acetyl-CoA:formate C-acetyltransferase.

Pyruvate formate lyase is found in the genus Enterobacteriaceae and some other organisms like algae but not in mammalia. It helps to regulate the anaerobic glucose metabolism. Using radical non-redox chemistry, the enzyme conducts the reversible conversion of pyruvate and coenzyme-A into formate and acetyl-CoA. The reaction occurs as depicted in the scheme below:

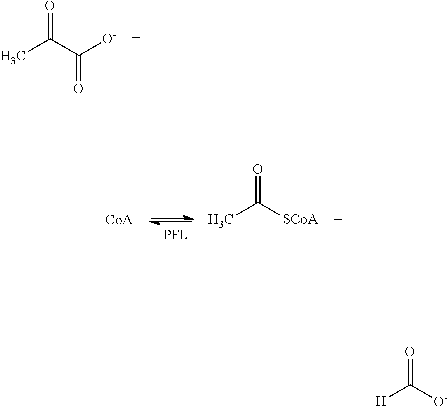

A person skilled in the art can readily recognize/identify a member of the pyruvate formate lyase family, particularly a member of an enterobacterial pyruvate formate lyase enzyme based on searching in GenBank or based on homology searching with SEQ ID NO: 1. SEQ ID NO: 1 corresponds with GenBank entry SL1344_0910 (also indicated as CBW17006.1), SEQ ID NO: 1 is the amino acid sequence derived from Salmonella enterica subsp. enterica serovar Typhimurium str. SL1344.

```
SEQ ID NO: 1
    1 mselneklat awegftkgdw qnevnvrdfi
        qknytpyegd esflagatda ttklwdsvme
  61 gvkqenrtha pvdfdtsvas titshdagyi
        nkalekivgl qteaplkrai ipfggikmve
121 gsckaynrel dpmikkifte yrkthnqgvf
        dvytpdilrc rksgvltglp daygrgriig
181 dyrrvalygi dylmkdkfaq ftslqsdlen
        gvnleatirl reeiaeqhra lgqikemaak
241 ygcdisgpat naqeaiqwty fgylaavksq
        ngaamsfgrv stfldayier dlkagkiteq
301 dagemidhlv mklrmvrflr tpeydelfsg
        dpiwatesig gmgvdgrtlv tknsfrflnt
361 lytmgpspep nitvlwsekl plnfkkfaak
        vsidtsslqy enddlmrpdf nnddyaiacc
421 vspmivgkqm qffgaranla ktmlyaingg
        vdeklkmqvg pksepikgdv Infdevmdrm
481 dhfmdwlakq yvtalnvihy mhdkysyeas
        Imalhdrdvi rtmacgiagl svaadslsai
541 kyakvkpird edglaidfei egeypqfgnn
        darvddmavd lverfmkkiq klttyrgaip
601 tqsvltitsn vvygkktgnt pdgrragapf
        gpganpmhgr dqkgavaslt svaklpfaya
661 kdgisytfsi vpnalgkdde vrktnlaglm
        dgyfhheasi eggqhlnvnv mnremlldam
721 ehpekypqlt irvsgyavrf nsltkeqqqd
        vitrtftqtm
```

Thus in a first embodiment the invention provides an inhibitor of the functional expression of an enterobacterial pyruvate formate lyase wherein the inhibitor is selected from an antisense oligonucleotide, a gapmer, a shRNA, a siRNA, a CRISPR, a TALEN, or a Zinc-finger nuclease.

In another embodiment the invention provides an inhibitor of the functional expression of an enterobacterial pyruvate formate lyase as depicted in SEQ ID NO: 1 and orthologous sequences of the genus Enterobacteriacea with an amino acid identity of the total length of SEQ ID NO: 1 of at least 60%, at least 70%, at least 80%, or at least 90% with SEQ ID NO: 1.

In yet another embodiment the invention provides an inhibitor of functional expression of pyruvate formate lyase as defined herein before for use as a medicament.

In yet another embodiment the invention provides an inhibitor of functional expression of pyruvate formate lyase as defined herein before for use to treat gut diseases where excessive apoptosis occurs.

In a particular embodiment, the application also provides an inhibitor of pyruvate release and/or pyruvate production for use to treat gut diseases where excessive apoptosis occurs.

Gut diseases (or gut conditions) which are known to have an excess of apoptosis in the gut cells are for example colitis, inflammatory bowel disease, chemotherapy induced mucositis or bacterial infections, such as food borne infections.

If inhibition is to be achieved at the DNA level, this may be done using gene therapy to knock-out or disrupt the target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genome of Enterobacteriacea. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway.

Gene inactivation, i.e. inhibition of functional expression of the gene, may for instance also be achieved through the creation of transgenic organisms expressing antisense RNA, or by administering antisense RNA to the subject. An antisense construct can be delivered, for example, as an expression plasmid, which, when transcribed in the cell, produces RNA that is complementary to at least a unique portion of the cellular PYRUVATE FORMATE LYASE.

A more rapid method for the inhibition of gene expression is based on the use of shorter antisense oligomers consisting of DNA, or other synthetic structural types such as phosphorothiates, 2′-0-alkylribonucleotide chimeras, locked nucleic acid (LNA), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack. An “antisense oligomer” refers to an antisense molecule or anti-gene agent that comprises an oligomer of at least about 10 nucleotides in length. In embodiments an antisense oligomer comprises at least 15, 18 20, 25, 30, 35, 40, or 50 nucleotides. Antisense approaches involve the design of oligonucleotides (either DNA or RNA, or derivatives thereof) that are complementary to an RNA encoded by polynucleotide sequences of PYRUVATE FORMATE LYASE. Antisense RNA may be introduced into a bacterial cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruitflies. RNA interference (RNAi) is a form of post-transcriptional gene silencing. The phenomenon of RNA interference was first observed and described in Caenorhabditis elegans where exogenous double-stranded RNA (dsRNA) was shown to specifically and potently disrupt the activity of genes containing homologous sequences through a mechanism that induces rapid degradation of the target RNA. Several reports describe the same catalytic phenomenon in other organisms, including experiments demonstrating spatial and/or temporal control of gene inactivation, including plant (Arabidopsis thaliana), protozoan (Trypanosoma bruceii), invertebrate (Drosophila melanogaster), and vertebrate species (Danio rerio and Xenopus laevis). The mediators of sequence-specific messenger RNA degradation are small interfering RNAs (siRNAs) generated by ribonuclease III cleavage from longer dsRNAs. Generally, the length of siRNAs is between 20-25 nucleotides (Elbashir et al. (2001) Nature 411, 494 498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. One or both strands of the siRNA of the invention can also comprise a 3′ overhang. A “3′ overhang” refers to at least one unpaired nucleotide extending from the 3′ end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′ deoxythymidine, is tolerated and does not affect the efficiency of RNAi degradation. In particular, the absence of a 2′ hydroxyl in the 2′ deoxythymidine significantly enhances the nuclease resistance of the 3′ overhang in tissue culture medium.

The siRNAs of the invention can be targeted to any stretch of approximately 19 to 25 contiguous nucleotides in any of the target PYRUVATE FORMATE LYASE RNA sequences (the “target sequence”), of which examples are given in the application. Techniques for selecting target sequences for siRNA are well known in the art. Thus, the sense strand of the present siRNA comprises a nucleotide sequence identical to any contiguous stretch of about 19 to about 25 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular intracellular environment.

As used herein, an “effective amount” of the siRNA is an amount sufficient to cause RNAi mediated degradation of the target mRNA, or an amount sufficient to inhibit the enterobacterial cell growth in a subject. RNAi mediated degradation of the target mRNA can be detected by measuring levels of the target mRNA or protein in the bacterial cells of a subject, using standard techniques for isolating and quantifying mRNA or protein as described above.

One skilled in the art can readily determine an effective amount of the siRNA of the invention to be administered to a given subject, by taking into account factors such as the size and weight of the subject; the extent of the disease penetration; the age, health and sex of the subject; the route of administration; and whether the administration is regional or systemic. Generally, an effective amount of the siRNA of the invention comprises an intracellular concentration of from about 1 nanomolar (nM) to about 100nM, preferably from about 2 nM to about 50 nM, more preferably from about 2.5 nM to about 10 nM. It is contemplated that greater or lesser amounts of siRNA can be administered.

Recently it has been shown that morpholino antisense oligonucleotides in zebrafish and frogs overcome the limitations of RNase H-competent antisense oligonucleotides, which include numerous non-specific effects due to the non-target-specific cleavage of other mRNA molecules caused by the low stringency requirements of RNase H. Morpholino oligomers therefore represent an important new class of antisense molecule. Oligomers of the invention may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

Another particularly form of antisense RNA strategy are gapmers. A gapmer is a chimeric antisense oligonucleotide that contains a central block of deoxynucleotide monomers sufficiently long to induce RNase H cleavage. The central block of a gapmer is flanked by blocks of 2′-O modified ribonucleotides or other artificially modified ribonucleotide monomers such as bridged nucleic acids (BNAs) that protect the internal block from nuclease degradation. Gapmers have been used to obtain RNase-H mediated cleavage of target RNAs, while reducing the number of phosphorothioate linkages. Phosphorothioates possess increased resistance to nucleases compared to unmodified DNA. However, they have several disadvantages. These include low binding capacity to complementary nucleic acids and non-specific binding to proteins that cause toxic side-effects limiting their applications. The occurrence of toxic side-effects together with non-specific binding causing off-target effects has stimulated the design of new artificial nucleic acids for the development of modified oligonucleotides that provide efficient and specific antisense activity in vivo without exhibiting toxic side-effects. By recruiting RNase H, gapmers selectively cleave the targeted oligonucleotide strand. The cleavage of this strand initiates an antisense effect. This approach has proven to be a powerful method in the inhibition of gene functions and is emerging as a popular approach for antisense therapeutics. Gapmers are offered commercially, e.g. LNA longRNA GapmeRs by Exiqon, or MOE gapmers by Isis pharmaceuticals. MOE gapmers or “2′MOE gapmers” are an antisense phosphorothioate oligonucleotide of 15-30 nucleotides wherein all of the backbone linkages are modified by adding a sulfur at the non-bridging oxygen (phosphorothioate) and a stretch of at least 10 consecutive nucleotides remain unmodified (deoxy sugars) and the remaining nucleotides contain an O′-methyl O′-ethyl substitution at the 2′ position (MOE).

According to a further aspect, the inhibitors of functional expression of PYRUVATE FORMATE LYASE are provided for use as a medicament.

The nature of the inhibitor is not vital to the invention, as long as it inhibits the functional expression of the PYRUVATE FORMATE LYASE gene. According to specific embodiments, the inhibitor is selected from an inhibitory RNA technology (such as a gapmer, a shRNA, a siRNA), a CRISPR, a TALEN, or a Zinc-finger nuclease or is a genetic inhibitor.

### Pannexin-1 Membrane Inhibition

Regulated mammalian cell death has a profound impact on tissue homeostasis and many pathological conditions. A long appreciated, yet elusively defined relationship exists between cell death and gastrointestinal pathologies with an underlying microbial component, but the direct impact of dying mammalian cells on bacterial growth is unclear. Here, using a combination of in vitro and in vivo approaches, we advance a concept that several Enterobacteriaceae, including patient-derived clinical isolates, have an efficient growth strategy to capitalize on soluble factors released from dying gut epithelial cells. Mammalian nutrients released after caspase-3/7-dependent apoptosis boosts the growth of three Enterobacteriaceae family members. The bacterial growth-promoting activity in apoptotic supernatants is observed using primary mouse colonic tissue, murine and human cell lines, multiple apoptotic triggers, and in conventional as well as germ-free mice in vivo. The present application is the first to show that inhibiting mammalian membrane pannexin-1 channels can be used to inhibit the overgrowth of Enterobacteriacea in the gut. Such overgrowth (or dysbiosis which is an equivalent term) occurs under conditions of apoptosis of gut cells such conditions are generally known as Inflammatory bowel disease (IBD), colitis, food borne infections and chemotherapy induced mucositis.

The Pannexin family consists of Pannexin-1, Pannexin-2, and Pannexin-3. The tissue distribution of Pannexin ranges from ubiquitous to very restrict areas depending on the paralog, and the distribution is often cell type-specific and/or developmentally regulated within some given tissues. Pannexin-1 is ubiquitously expressed in human tissues, such as the brain, heart, lung, liver, small intestine, pancreas, spleen, colon, skeletal muscle, skin, testis, ovary, placenta, thymus, prostate, blood endothelium, and erythrocytes. Pannexin-1 is also found to be expressed in the central nervous system including the cerebellum, cortex, lens, retina, pyramidal cells, interneurons of the neocortex and hippocampus, substantia nigra, amygdala, olfactory bulb, neurons, and glial cells. The expression of Pannexin-2 is more restricted to the central nervous system, including the cerebellum, cerebral cortex, occipital pole frontal lobe, medulla, temporal lobe, and putamen. However, low expression of Pannexin-2 is also found in thyroid, kidney, and liver tissues. Pannexin-2 protein expression is further identified in the basal cells of the stria vascularis and spiral ganglion neurons of the rat cochlear system. Pannexin-3 is found to be expressed in osteoblasts, synovial fibroblasts, whole joints of mouse paws, and cartilage from the inner ear. Pannexin-3 is also expressed in many cultured cell lines.

Several medicines are on the market known to have as a mechanism of action to inhibit membrane pannexin-1 channels.

Spironolactone, sold under the brand name Aldactone among others, is a medication that is primarily used to treat fluid build-up due to heart failure, liver scarring, or kidney disease. It is also used in the treatment of high blood pressure, low blood potassium that does not improve with supplementation, early puberty in boys, acne and excessive hair growth in women, and as a part of transgender hormone therapy in transgender women. Spironolactone is usually taken orally. Interestingly spironolactone is also described as a membrane pannexin-I channel inhibitor (see Good ME et al (2018)Circ. Res. 122 (4): 606-615).

Tenofovir disoproxil (a prodrug of Tenofovir), sold under the trade name Viread among others, is a medication used to treat chronic hepatitis B and to prevent and treat HIV/AIDS. The drug is off patent and is orally bioavailable. Another prodrug of Tenofovir is known as tenofovir alafenomide. In the present invention tenofovir is used to refer to the two different prodrugs. Tenofovir is described in literature as an agent to inhibit membrane pannexin-1 channels (see Feig JL et al (2017) PLOS One 12(11): e0188135.

Probenecid, also sold under the brand name Probalan, is a medication that increases uric acid excretion in the urine. It is primarily used in treating gout and hyperuricemia. Probenecid is also described to act as a membrane pannexin-1 channel inhibitor (see Silverman W et al (2008) Am J Physiol Cell Physiol. 295(3):C761-7.

Trovafloxacin (sold as Trovan and Turvel which are the brand names) is a broad-spectrum antibiotic that inhibits the uncoiling of supercoiled DNA in various bacteria by blocking the activity of DNA gyrase and topoisomerase IV. It was withdrawn from the market due to the risk of hepatotoxicity. It had better gram-positive bacterial coverage and less gram-negative coverage than the fluoroquinolones. Trovafloxacin is described as a membrane pannexin-1 channel inhibitor (see Poon IKH et al (2014) Nature 507(7492):329-34).

Known pannexin-1 channel inhibitors can be repurposed for the treatment of gut diseases with an excess of apoptosis with an aim to correct the excessive overgrowth of Enterobacteriacea.

In one embodiment the present invention provides an inhibitor of the membrane channel pannexin-I for use to treat gut diseases where excessive apoptosis occurs.

In a particular embodiment gut diseases are for example colitis, inflammatory bowel disease, chemotherapy induced mucositis or bacterial infection.

In another particular embodiment the bacterial infection is due to a food-borne infection.

In another particular embodiment the invention provides spironolactone, tenofovir, probenecid or trovafloxacin for the treatment of gut diseases where excessive apoptosis occurs.

### Dose and Administration of Pannexin-1 Membrane Channel Inhibitors

Based upon standard laboratory techniques known to evaluate compounds useful for the treatment of diseases cited herein, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of the indications cited herein. The amount of the active ingredient to be administered in the treatment can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 50 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight. The average daily oral dosage regimen will preferably be from 0.01 to 100 mg/kg of total body weight. The average daily intrathecal dosage regimen will preferably be from 0.01 to 100 mg/kg of total body weight.

It is evident for the skilled artisan that the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

To summarize, the present invention provides subject-matter as set forth in any one and all of (1) to (6) below:
(1) An inhibitor of the pannexin-I membrane channel for use to treat gut diseases where excessive apoptosis occurs.
(2) An inhibitor for use according to (1) wherein the gut disease is associated with colitis, inflammatory bowel disease, chemotherapy induced mucositis or bacterial infection.
(3) An inhibitor for use according to (2) wherein the bacterial infection is an enterobacterial infection.
(4) An inhibitor for use according to (3) wherein the enterobacterial infection is a food-borne infection.
(5) An inhibitor for use according to (4) wherein the food-borne infection is due to Klebsiella species, Salmonella species or Escherichia species
(6) An inhibitor for use according to any one of (1) to (5) wherein the pannexin-I membrane channel inhibitor is selected from spironolactone, tenofovir, probenecid and trovafloxacin.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Caspase-Dependent Apoptosis of Primary Colonocytes Promotes Salmonella Growth

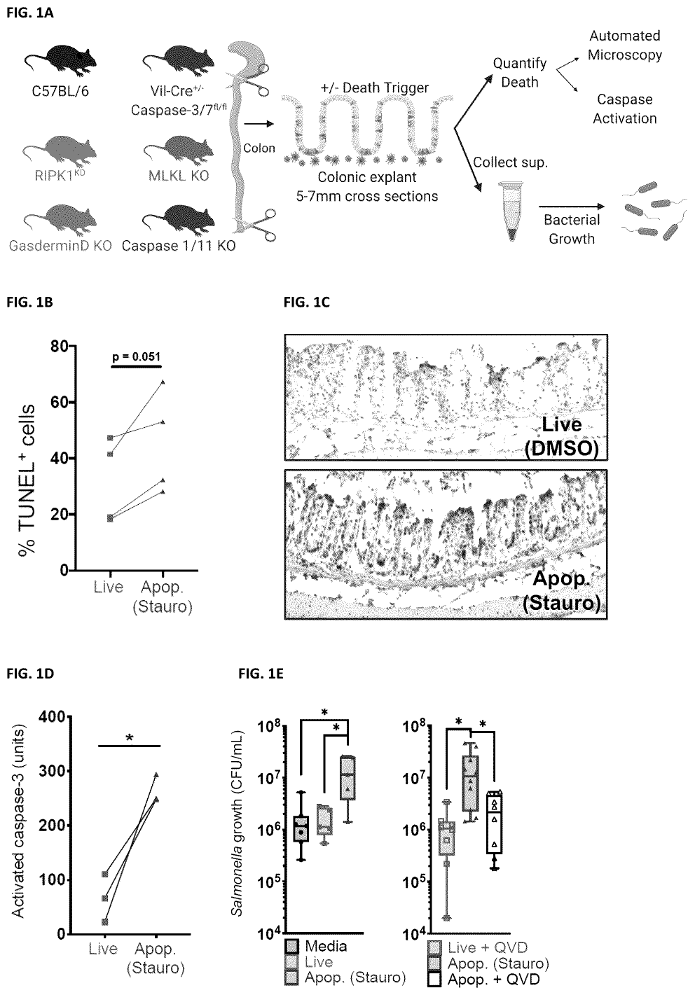

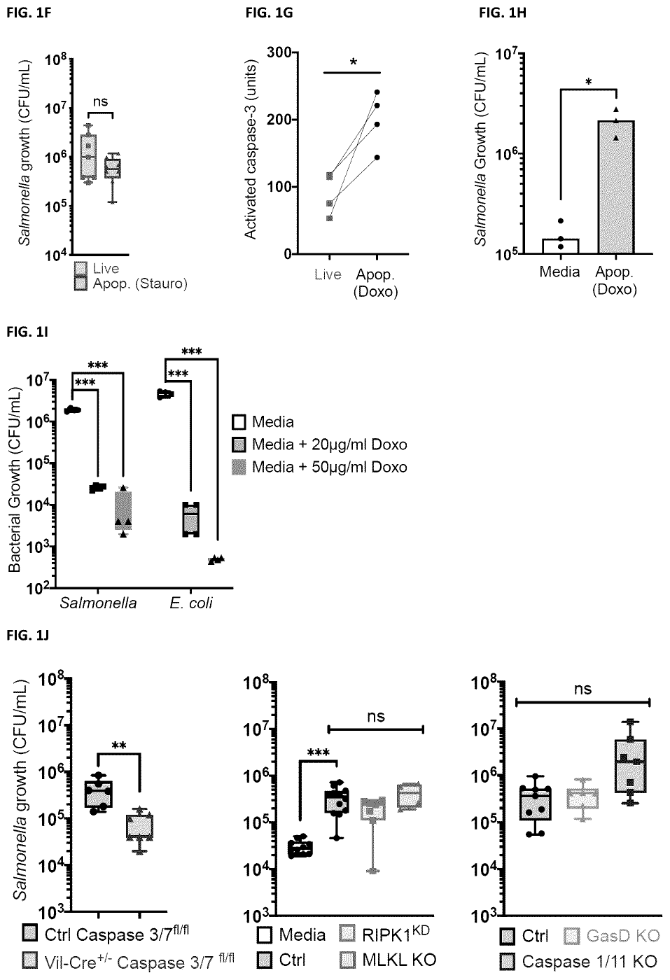

As a first test of the hypothesis that death-dependent soluble factors released from mammalian cells could augment bacterial growth, we established primary colonocyte culture explants and induced apoptotic cell death via staurosporine treatment (see schematic in FIG. 1A). The colonic explants maintained general tissue structure, including villi and the underlying lamina propria (data not shown). After verifying primary colonocyte cell death via TUNEL staining (FIG. 1B), cleaved caspase-3 staining (FIG. 1C), and caspase activation assays (FIG. 1D), we tested the apoptotic supernatants for their ability to induce Salmonella growth. Compared to live (vehicle-treated) colonocyte supernatants, the staurosporine-treated apoptotic supernatants induced ˜10-fold greater Salmonella growth (FIG. 1E, left). Notably, the increased Salmonella growth seen in dying primary colonocyte supernatants compared to fresh media controls suggests that dying cells actively ‘secrete’ factors into the media that enhance bacterial growth, rather than living cells simply consuming nutrients from the growth medium. Enhanced Salmonella growth in dying colonocyte supernatant was dependent on caspase activation, as the pan-caspase inhibitor QVD restricted Salmonella growth to live supernatant control levels (FIG. 1E, right). The primary colonic explant system contained non-epithelial cells, including CD45⁺ myeloid cells; yet, the total CD45⁺ fraction as well as the CD45⁺TUNEL⁺ populations did not increase following staurosporine treatment (data not shown). To more directly test caspase-dependent epithelial cell apoptosis, we utilized colonic explants from Vil-Cre^{+/-} Caspase-3/7^{fl/fl} mice, in which caspase 3 and caspase 7 are both deleted specifically in the epithelial cell compartment via villin-Cre²¹. In contrast to control C57BL/6 mice (FIG. 1E), staurosporine-treated Vil-Cre^{+/-} Caspase-3/7^{fl/fl} colonocytes failed to enhance Salmonella growth above live colonocyte controls (FIG. 1F). We observed similar effects when we treated colonic explants with the cytotoxic chemotherapeutic doxorubicin (Doxo). Doxorubicin treatment induced caspase-3 activation (FIG. 1G), and these supernatants directly enhanced Salmonella outgrowth (FIG. 1H). This is particularly striking, as bacterial outgrowth occurs despite the direct antimicrobial action of doxorubicin on Enterobacteriaceae (FIG. 1I). Doxorubicin-induced Salmonella outgrowth was dependent on executioner caspases-3 and 7 within intestinal epithelial cells (FIG. 1J left), suggesting a requirement for apoptosis. As recent studies have highlighted the interconnectivity of different regulated cell death pathways^{22,23}, we also tested mice lacking specific components of the necroptotic or pyroptotic machinery. In contrast to apoptosis-deficient colonocytes, doxorubicin-treated colonic explants from mice defective in necroptotic cell death, including RIPK1^{kinase dead} and MLKL^{KO} did not restrict Salmonella growth compared to littermate controls (FIG. 1J center); similarly, doxorubicin treatment of colonocytes from mice lacking the pyroptotic executioners gasdermin D and caspase-1/11 also did not restrict enhanced Salmonella growth (FIG. 1J right). These findings demonstrate the necessity and specificity for caspase-dependent intestinal epithelial cell apoptosis in promoting Salmonella growth in these primary colonic explant supernatants.

### Example 2. Regulated Mammalian Cell Death Enhances Enterobacteriaceae Growth

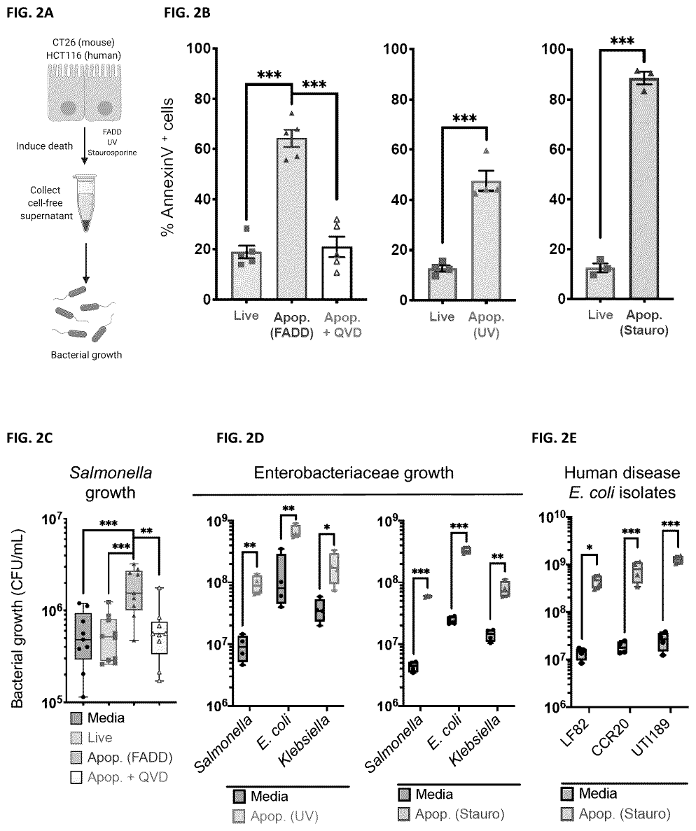

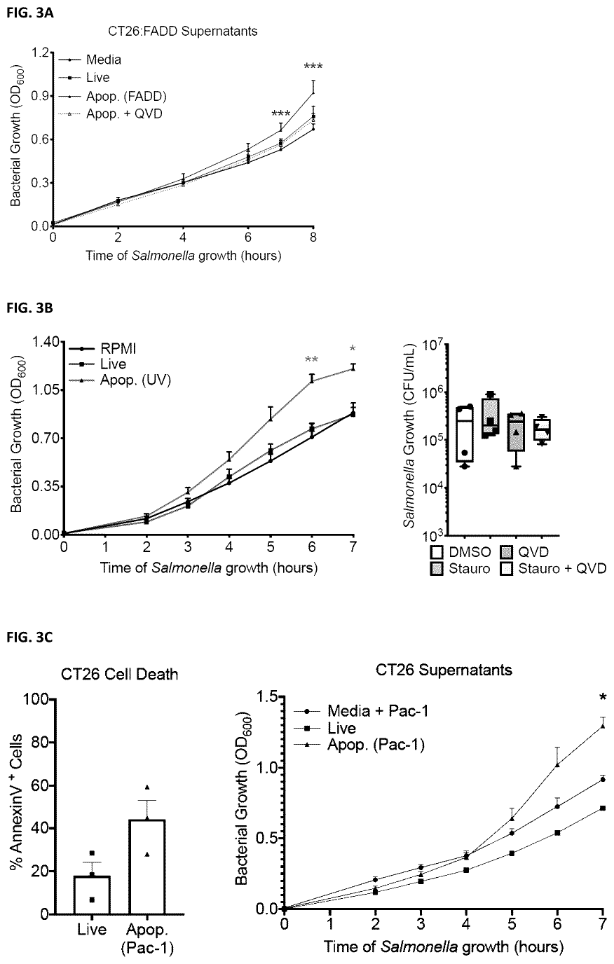

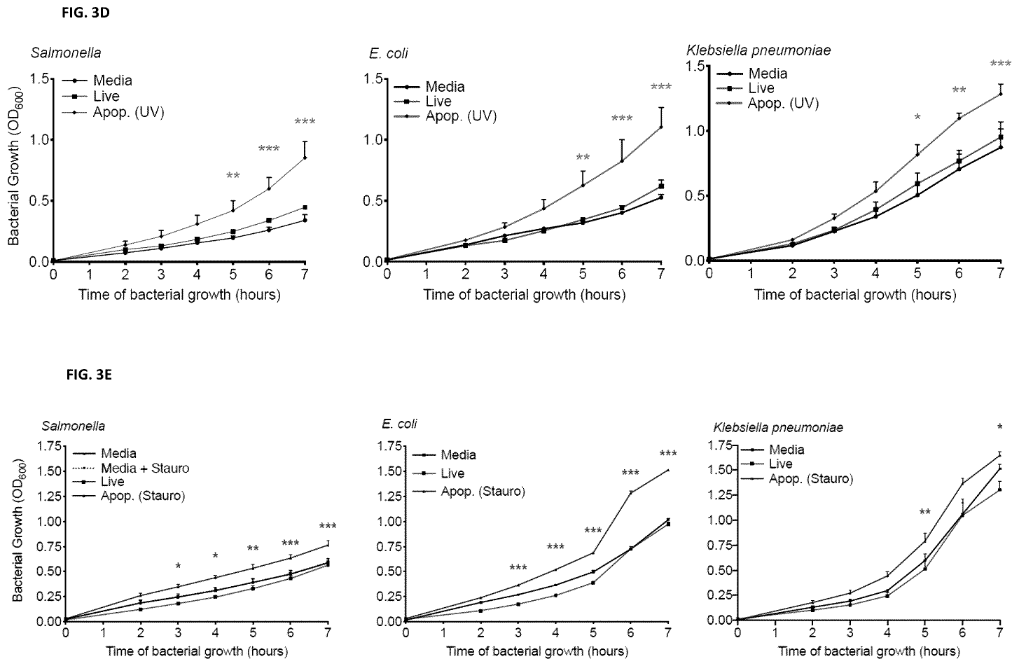

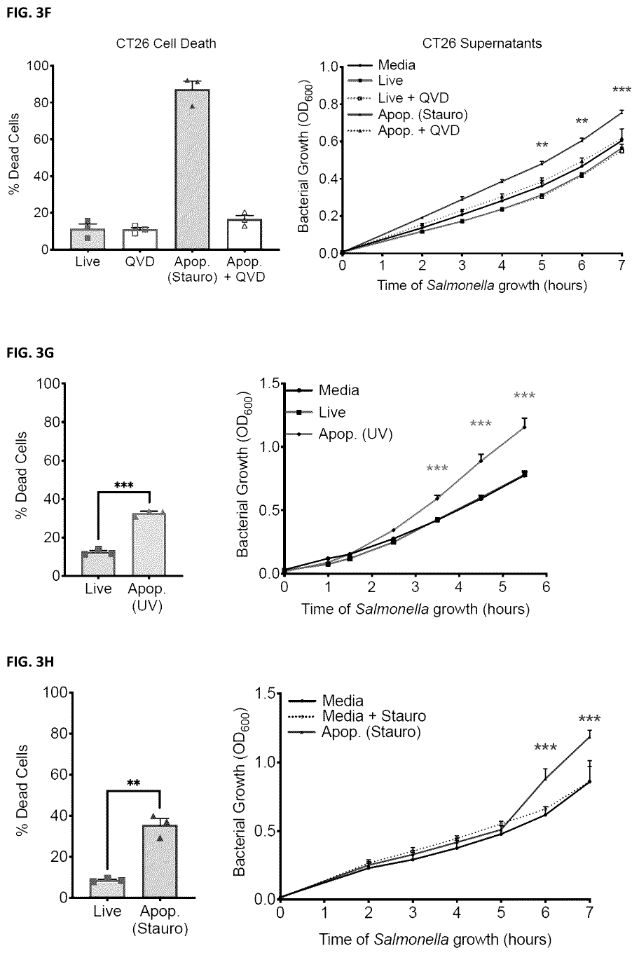

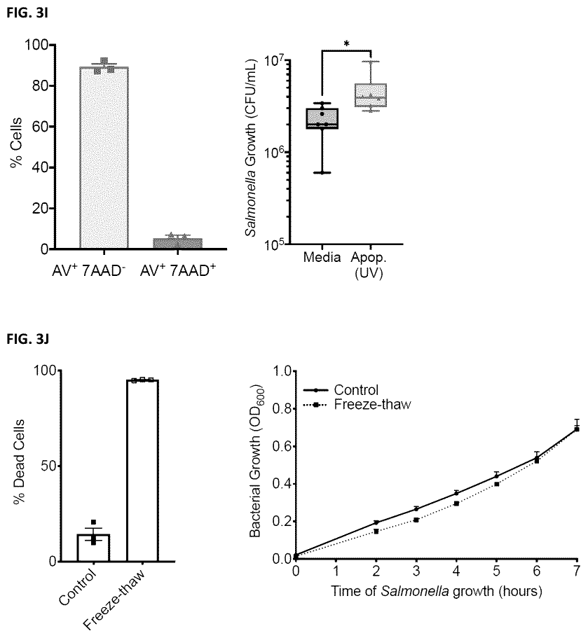

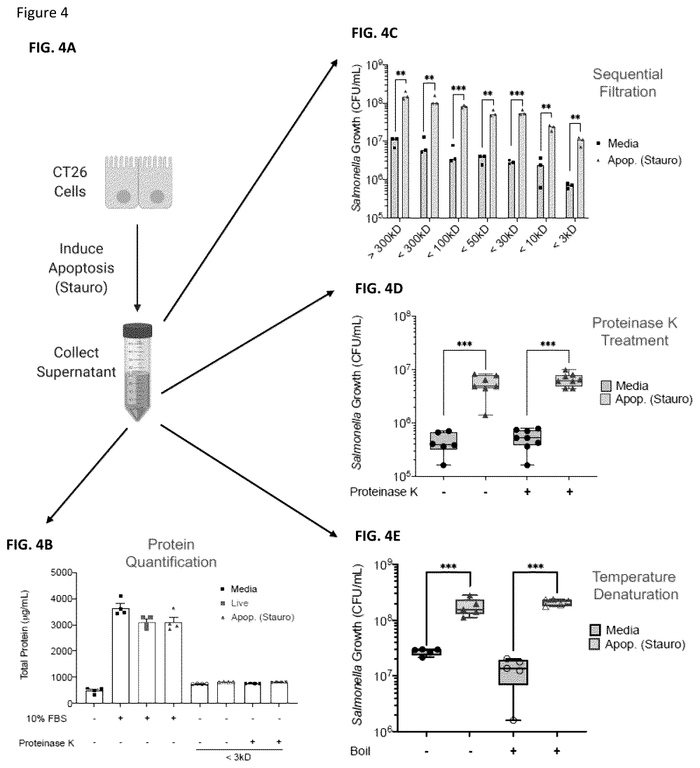

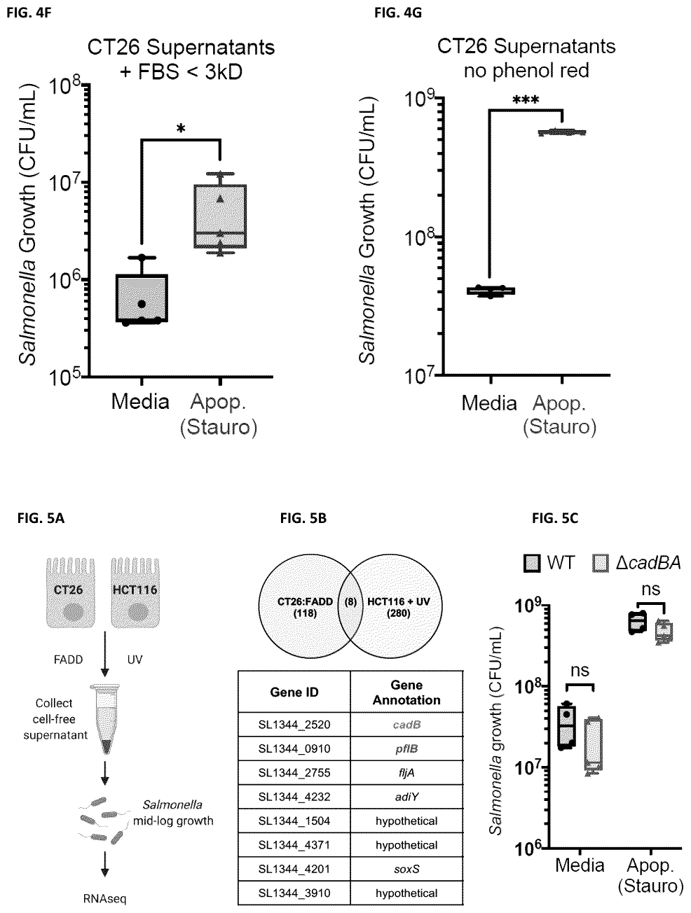

To further investigate the details of intestinal epithelial cell apoptosis in promoting enhanced bacterial growth, we turned to epithelial cell lines. We induced apoptotic cell death using different apoptotic stimuli in mouse or human colonic epithelial cell lines, collected the cell-free supernatants, and tested them in bacterial growth studies (see schematic in FIG. 2A). First, we used murine colonic CT26 cells that have been transformed with a doxycycline-inducible FADD-dimerizing construct (CT26:FADD)²⁴, a tightly controlled in vitro system of caspase-dependent apoptotic cell death and confirmed increased cell death of CT26:FADD cells (FIG. 2B, left). The supernatant from apoptotic CT26:FADD cells promoted a significant increase in Salmonella growth compared to supernatants from untreated live cell controls or fresh media (FIG. 2C). Increased Salmonella growth required caspase activity, as treating CT26:FADD cells with the pan-caspase inhibitor QVD reduced cell death (FIG. 2B, left) and apoptotic caspase activation (data not shown), and reduced Salmonella growth back to media and live cell supernatant controls (FIG. 2C). For two additional modes of apoptosis induction, we induced apoptosis in wild type CT26 cells with either UV irradiation or staurosporine (FIG. 2B, center and right), and again the supernatants of these apoptotic cells strongly induced Salmonella growth compared to control treatments (FIG. 2D). The ability of apoptotic mammalian cell supernatants to promote bacterial growth was not restricted to Salmonella, as dying CT26 cell supernatants also enhanced the growth of additional members of the Enterobacteriaceae family, including a human intestinal commensal strain of Escherichia coli²⁵ (E. coli) and the intestinal commensal but systemic pathogen Klebsiella pneumoniae²⁶ (Klebsiella) (FIG. 2D). Importantly, several clinical isolates of E. coli displayed similar enhanced growth in apoptotic supernatant. Enhanced growth was observed in E. coli isolates obtained from patients with Crohn's Disease (LF82²⁷), colorectal cancer (CCR20²⁸), and urinary tract infection (UTI189²⁹) (FIG. 2E). Multiple analyses established that the enhanced bacterial growth was a direct effect of cell death components released in the supernatants of apoptotic CT26 cells, including: (i) increased bacterial growth was seen over time for several members of the Enterobacteriaceae family (FIG. 3A,D,E); (ii) bacterial growth was not influenced by mammalian culture media selection or the death triggers themselves (FIG. 3B); (iii) enhanced bacterial growth was observed with supernatants from cells treated with the caspase-3 activator Pac-1 (FIG. 3C), and (iv) the growth effect was stunted by the addition of mammalian caspase inhibitors (FIG. 2C; FIG. 3A,F). Similar results were obtained using the human colonic epithelial cell line HCT116 in response to both UV and staurosporine-induced cell death (FIG. 3G-H). Colonic cell lines such as CT26 and HCT116 display heterogeneity in their death profile for membrane permeability (data not shown), while Jurkat cells, an immortalized human T lymphocyte cell line, display more uniform apoptotic cell death without secondary necrosis in response to UV irradiation (data not shown). Apoptotic Jurkat supernatants stimulated increased Salmonella growth (FIG. 3I), suggesting that factors involved in promoting bacterial growth are conserved across distinct mammalian cell types, and are less dependent on membrane permeability or secondary necrosis. Strikingly, forced necrosis by repeated freeze-thaw cycles without an apoptotic trigger completely lysed CT26 cells but did not enhance Salmonella growth (FIG. 3J), indicating the requirement of regulated apoptosis in this process. We then asked what type of factors within apoptotic cell supernatants promoted bacterial growth, and a multitude of approaches suggested that proteins do not play a role (FIG. 4A). First, in agreement with previous findings¹⁶, supernatants from apoptotic cells contained similar levels of total protein as untreated live cell supernatants (FIG. 4B). Second, a rigorous sequential filtration strategy, to remove extracellular vesicles as well as the majority of supernatant proteins, demonstrated that soluble factors <3 kDa in size in the apoptotic supernatants were sufficient to significantly enhance Salmonella growth (FIG. 4C). Third, this filtration strategy coupled to proteinase K treatment produced similar results (FIG. 4D). Finally, the released mammalian factors enhancing Salmonella growth were insensitive to temperature denaturation (FIG. 4E), independent of serum (FIG. 4F), and independent of phenol red in the culture media (FIG. 4G). Collectively, these data suggested that small molecules or metabolites <3 kDa in size released from apoptotic colonocytes act as nutrients that augment pathogenic (Salmonella and E. coli LF82/CCR20/UTI189), commensal (E. coli HS), and opportunistic (Klebsiella) Enterobacteriaceae growth; we term this process “death-induced nutrient release” (DINNR).

### Example 3. DINNR Induces a Specific Transcriptional Response in Salmonella

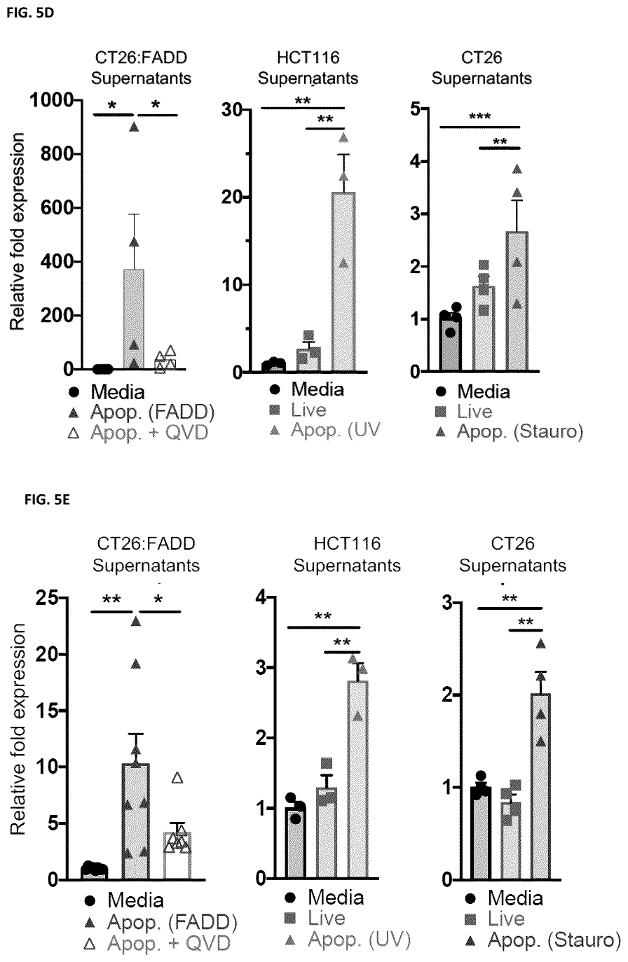

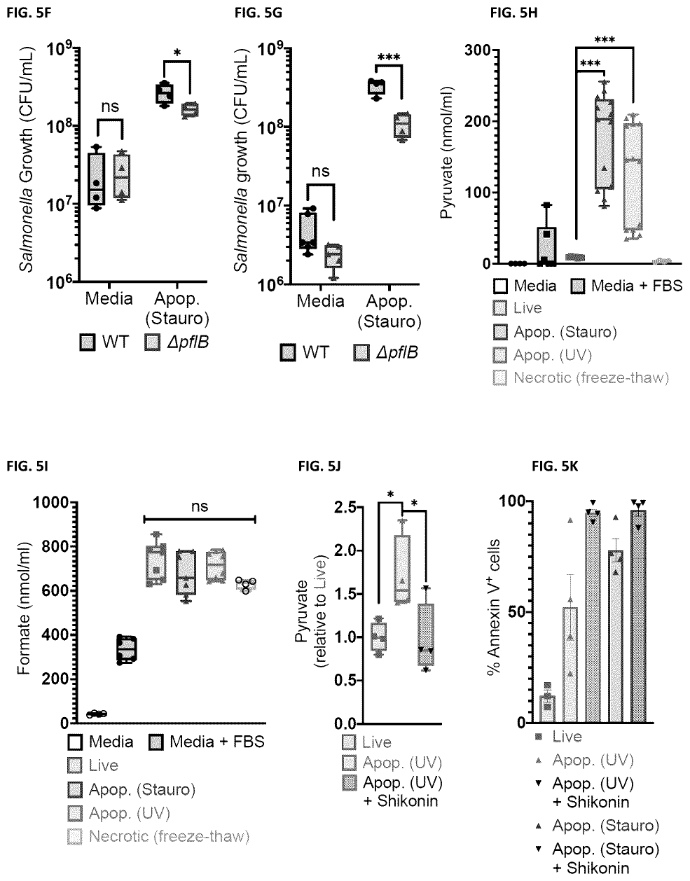

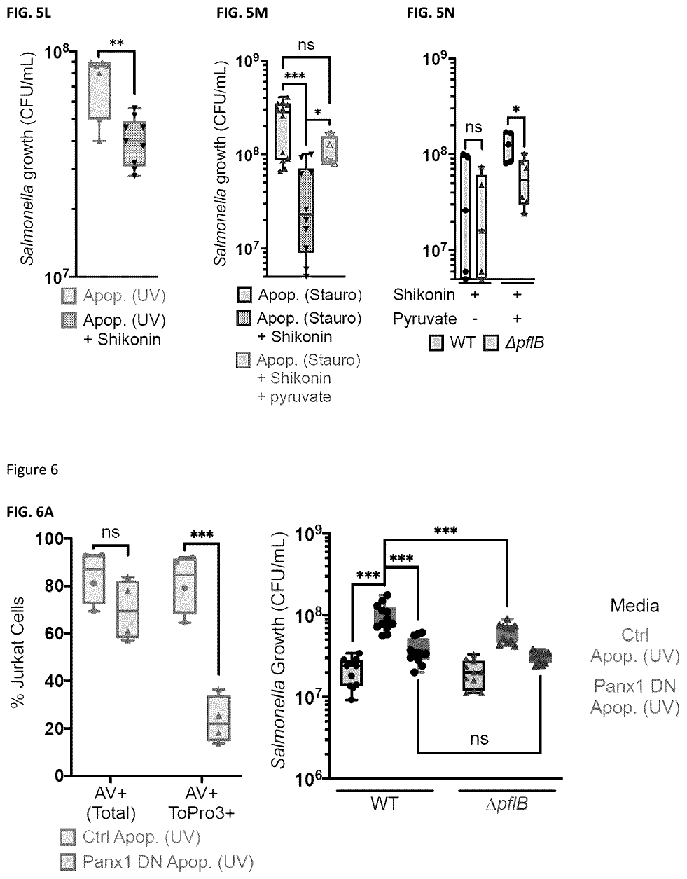

To address how the mammalian apoptotic nutrients may promote bacterial growth, we chose to focus on pathogenic Salmonella as our model organism. We performed RNAseq analysis of Salmonella grown to mid-logarithmic growth phase (which immediately preceded a growth phenotype) in apoptotic supernatants, fresh media, or live cell supernatant controls (schematically shown in FIG. 5A). To maximize the likelihood of identifying biologically significant target genes, we used two different mammalian cell lines (CT26 and HCT116), two different triggers of apoptosis (FADD and UV, respectively), and we focused our analyses on differentially regulated Salmonella genes that had a log₂ fold-change with an absolute value of >1.0. We identified 126 differentially regulated Salmonella genes using the CT26:FADD system and 288 genes in the HCT116 system (FIG. 5B).

Comparing the CT26 and HCT116 datasets narrowed the list to eight Salmonella genes that were similarly regulated between the two systems (FIG. 5B). Within these eight Salmonella genes, there were two target genes with a high potential to directly contribute to enhanced bacterial growth in dying mammalian cell supernatants. The first, cadB (SL1344_2520), encodes a lysine/cadaverine antiporter that is involved in bacterial cytosolic pH regulation together with the co-transcribed lysine decarboxylase CadA³⁰. The second, pflB (SL1344_0910), is a pyruvate formate-lyase that produces Acetyl-CoA and formate from pyruvate³¹. We chose to focus on cadB and pflB rather than the remaining six target genes for the following rationale: three are annotated as genes of unknown function, and three additional genes (fljA, adiY, soxS) are annotated as transcriptional regulators that could indirectly influence this phenotype (via regulation of multiple other genes). Apoptotic supernatants derived from both CT26 and HCT116 cells using the FADD, staurosporine, and UV triggers significantly increased the expression of cadB (FIG. 5D) and pflB (FIG. 5E) in Salmonella (via qPCR). To test the functional relevance of cadB and pflB gene upregulation, we constructed deletion strains of Salmonella. Deletion of the cadBA operon did not impact growth in apoptotic supernatants (FIG. 5C). In contrast, growth of the ΔpflB strain was partially, but significantly, reduced in apoptotic supernatants under both aerobic (FIG. 5F) and anaerobic growth conditions in vitro (FIG. 5G). These findings suggest that PflB directly contributes to, but is not solely responsible for, Salmonella growth in apoptotic supernatants.

Since PflB is a pyruvate formate lyase, we next tested whether apoptotic cells released either of the metabolites associated with PflB function. CT26 cells triggered with either staurosporine or UV irradiation released significantly higher levels of pyruvate compared to live cell supernatants or fresh media controls (FIG. 5H). Importantly, cells induced to die via unregulated necrosis did not release significant amounts of pyruvate (FIG. 5H) suggesting that pyruvate synthesis and release during cell death is likely a regulated process. In contrast to pyruvate, there was no difference in formate concentrations within the supernatants of living, apoptotic, or necrotic cells (FIG. 5I). To test whether mammalian-produced pyruvate was necessary for Salmonella growth, we treated CT26 cells with the pyruvate kinase M2 (PKM2) inhibitor Shikonin³². After confirming that treatment of apoptotic cells with Shikonin significantly reduced the amount of secreted pyruvate (FIG. 5J) without inhibiting cell death (FIG. 5K), we tested these supernatants for enhanced Salmonella growth. Strikingly, treatment of mammalian cells with Shikonin significantly reduced the growth of WT Salmonella (FIG. 5L). Further, PflB-dependent Salmonella growth was ablated in apoptotic supernatants from cells treated with Shikonin (FIG. 5N), which could be rescued by add-back of exogenous pyruvate (FIG. 5M). Together, these results identify mammalian-produced pyruvate during apoptosis as a direct contributor to PflB-dependent growth in Salmonella.

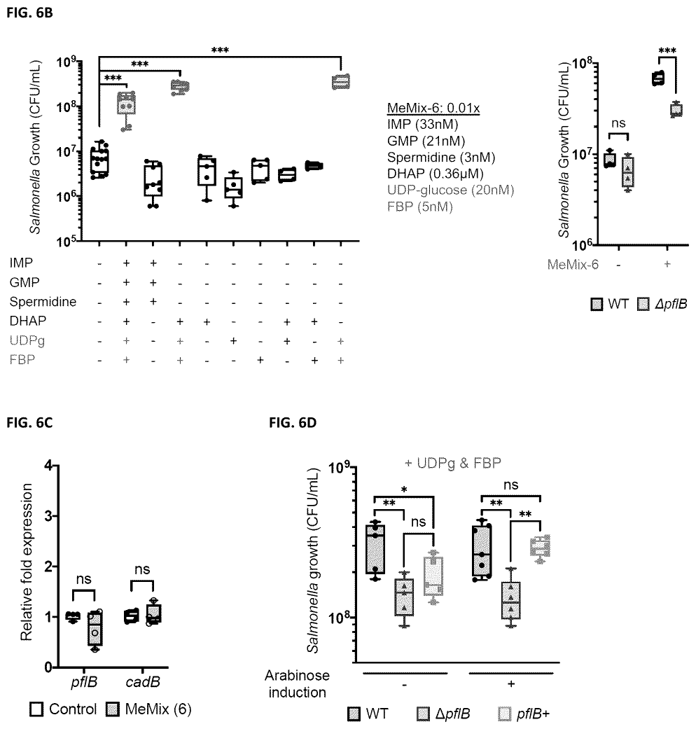

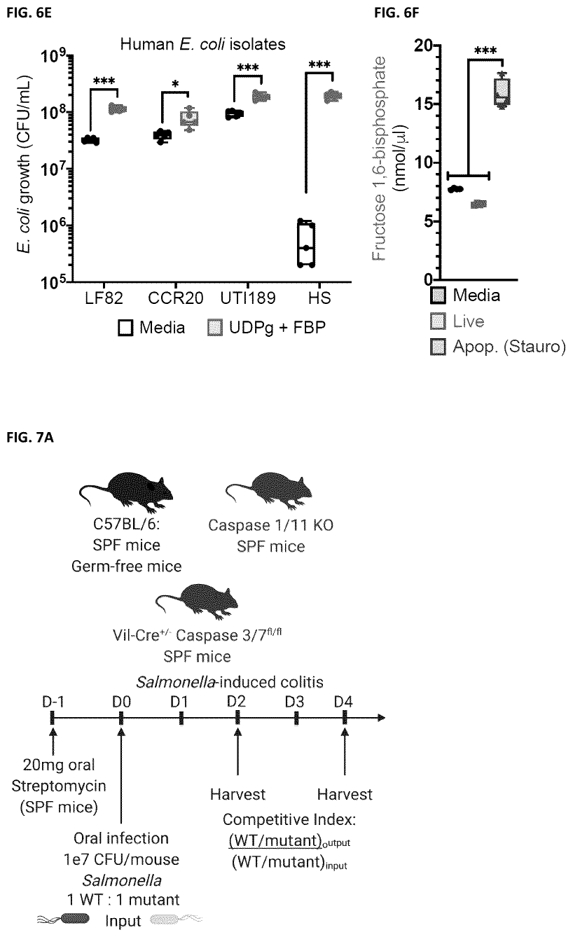

Recently, a group of six metabolites (inosine monophosphate (IMP), guanosine monophosphate (GMP), spermidine, dihydroxyacetone phosphate (DHAP), UDP-glucose, and fructose 1,6-bisphosphate) was shown to be released from dying mammalian cells (across cell types) in response to a variety of apoptotic triggers¹⁹. These six metabolites, whose release requires the Pannexin-1 (Panx1) membrane channel, have profound effects on mammalian intercellular communication¹⁹. We next asked whether Enterobacteriaceae can utilize these metabolites for bacterial growth. As a first test of this hypothesis, we utilized Jurkat cells expressing a dominant negative form of the Panx1 channel (‘Panx1-DN’) that have a mutation at the Panx1 caspase cleavage site³³. These cells undergo apoptosis comparable to control Jurkat cells, but fail to open the Panx1 channel (as measured by TOPRO-3 dye uptake)¹⁹ (FIG. 6A, left). Interestingly, growth of WT Salmonella in apoptotic supernatants from Panx1-DN expressing cells not able to open the Panx1 channel is significantly blunted compared to apoptotic supernatants from control cells (FIG. 6A, right). Additionally, PflB-driven growth is ablated specifically in apoptotic Panx1-DN supernatants (FIG. 6A) suggesting a link between Panx1-dependent metabolites and Salmonella PflB. In a proof-of-principle experiment, we tested a mixture of the six Panx1-dependent metabolites (MeMix⁶) on bacterial growth. MeMix⁶ was sufficient to potently promote Salmonella growth when added to media at concentrations detected in apoptotic supernatants¹⁹ (FIG. 6B). In subsequent experiments testing the necessity for individual metabolites, the specific combination of two metabolites—UDP-glucose (UDPg) and fructose 1,6-bisphosphate (FBP)—was sufficient to promote robust outgrowth at levels similar to MeMix⁶ (FIG. 6B, left). As with the Panx1-DN supernatants, growth of the pflB mutant was significantly blunted in the presence of the purified metabolites (FIG. 6B). The requirement for PflB in these assays was further confirmed by re-expressing pflB in trans in the ΔpflB strain (FIG. 6D). Finally, pathogenic and commensal strains of E. coli displayed similar enhanced growth in the presence of Panx1-dependent metabolites (FIG. 6E), highlighting a conserved utilization of these metabolites across members of the Enterobacteriaceae. We could also detect the release of fructose 1,6-bisphosphate by apoptotic epithelial cells (FIG. 6F). Collectively, these data identify a core Salmonella transcriptional program induced by mammalian apoptotic supernatants that is conserved across three triggers of apoptosis (FADD, UV, staurosporine) and two colonic cell line systems (CT26, HCT116), and reveal PflB as an important component for promoting bacterial growth.

Mechanistically, Salmonella utilizes mammalian-derived pyruvate and can exploit mammalian Panx1-dependent apoptotic metabolites for growth, which is moreover observed with multiple species of Enterobacteriaceae.

### Example 4. Death Responsive Genes Drive Salmonella Colonization in Vivo

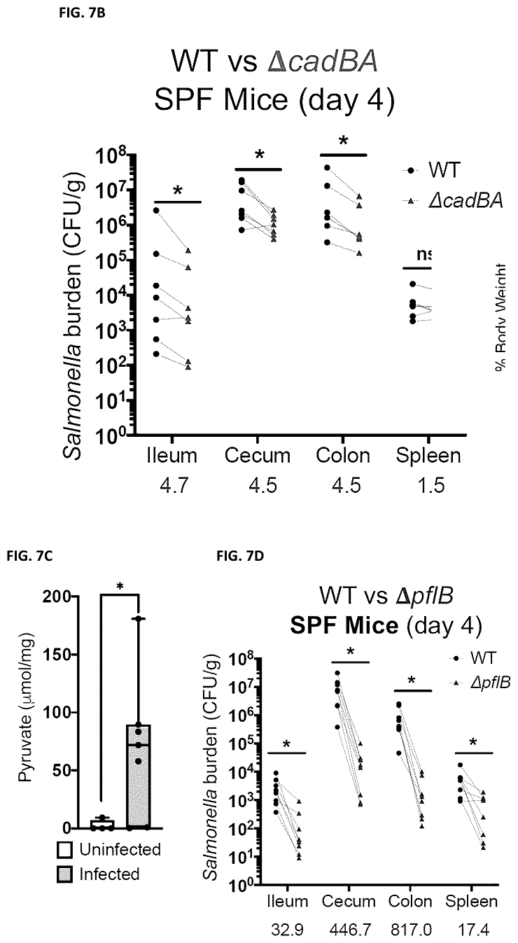

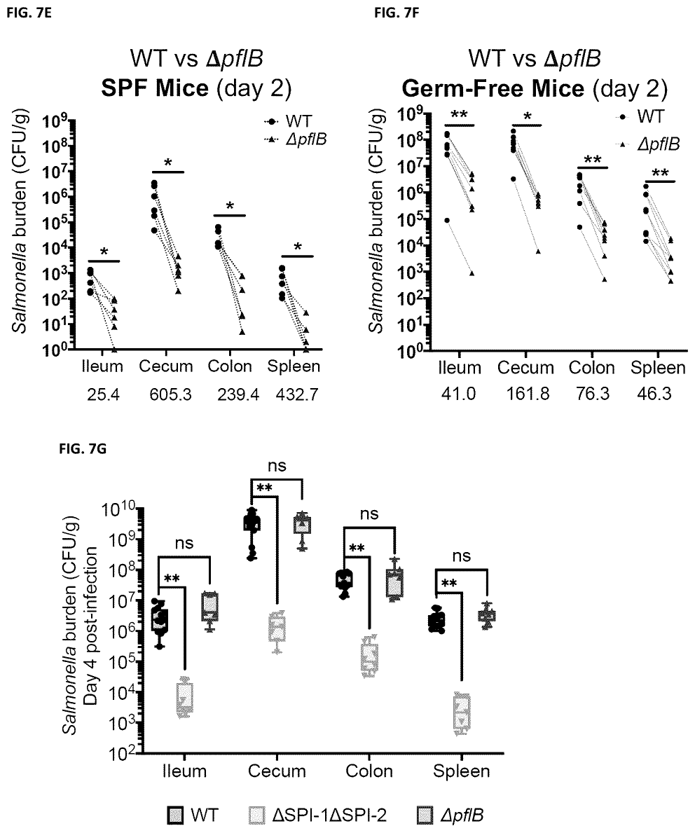

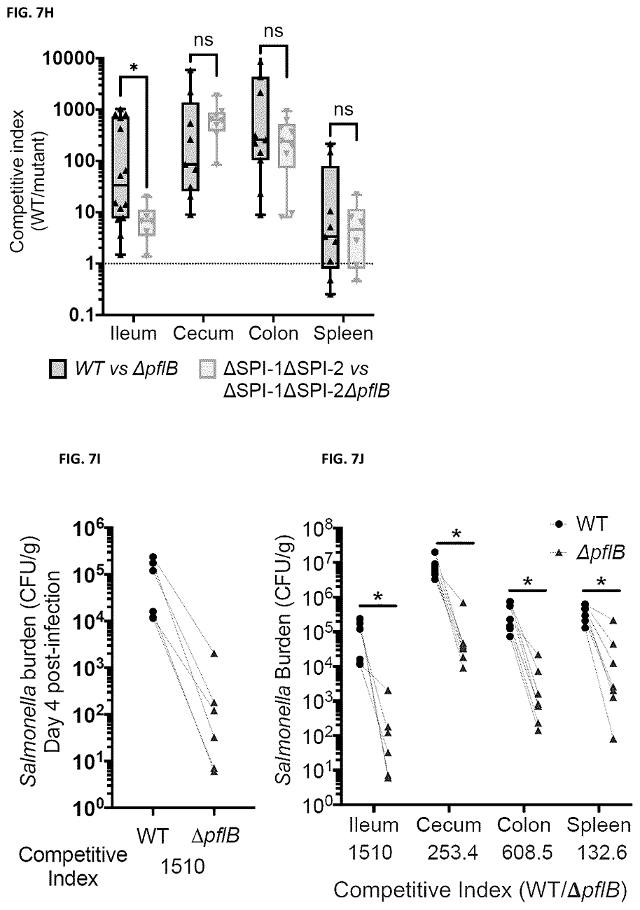

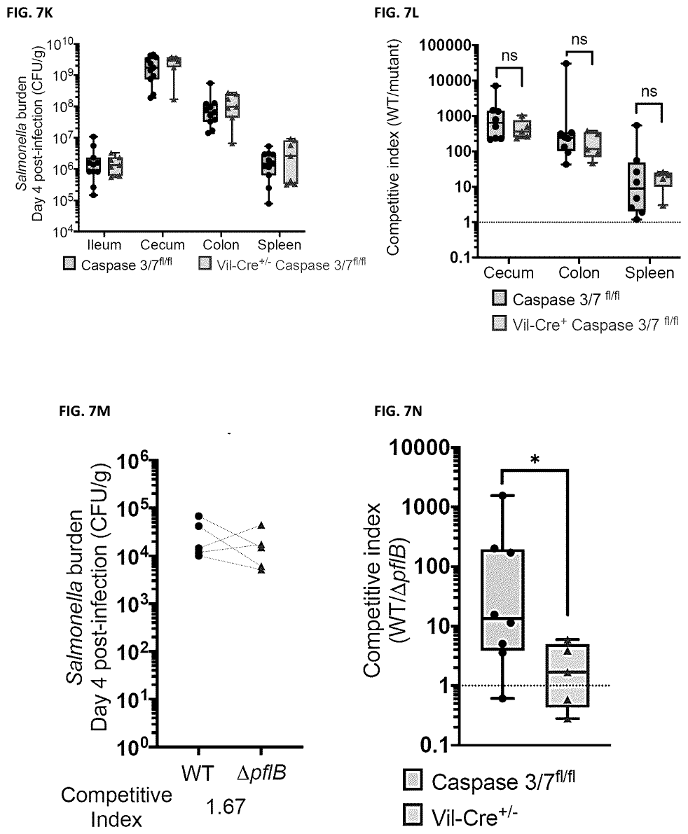

To test the relevance of the cadBA operon and pflB in a model of foodborne Salmonella infection, we utilized the streptomycin pretreatment mouse model³⁴. These mice display enterocolitis and diarrheal disease that recapitulates several aspects of the clinical presentation in Salmonella-infected patients and other experimental animal models³⁴. During infection, Salmonella burden is heavily influenced by the resident microbiota³⁴, colonocyte-dependent factors³⁵, and intestinal inflammation³⁶ in a tissue-specific manner. Salmonella utilizes two type-3 secretion systems (T3SS) encoded within the Salmonella Pathogenicity Island (SPI) 1 and SPI-2 that are largely responsible for the inflammation and morbidity in this model³⁴. To test the importance of our RNAseq-identified Salmonella genes, we employed competitive infections wherein mice are infected with equal numbers of WT and mutant strains of Salmonella, and then the relative ratio (i.e. colonization fitness) of each Salmonella strain is compared (schematically shown in FIG. 7A). The ΔcadBA strain was significantly less fit than WT Salmonella within the intestinal tract, with a median 5-fold enrichment of the WT Salmonella strain in the ileum, cecum, and colon relative to the ΔcadBA strain (FIG. 7B) (consistent with a previous study on the CadBA system in Salmonella in vivo³⁷). When we measured the concentrations of luminal pyruvate, we noted a significant increase in infected mice compared to uninfected controls (FIG. 7C), suggesting a potential requirement for PflB. Indeed, the fitness deficiency of the ΔpflB strain was striking when compared to WT Salmonella (FIG. 7D), with an 800-fold difference in the colon. The larger in vivo phenotype observed with the ΔpflB strain compared to in vitro settings could be related to increased pflB-activating signals or a stronger reliance on this pathway in the complex in vivo environment relative to defined culture media in vitro. It has been suggested that Salmonella may utilize microbiota-produced products in vivo, rather than rely on PflB³⁸. To test this, we repeated WT vs ΔpflB competitive infections in SPF and germ-free mice but analyzed the animals on day 2, as the germ-free mice were hypersensitive to infection and the experiment had to be stopped. Even at this earlier stage of infection, WT Salmonella was significantly more fit than the ΔpflB strain throughout all tissues in both SPF mice and germ-free mice (FIG. 7E-F). Importantly, while the level of WT Salmonella median enrichment was similar in the ilea of SPF and germ-free mice; the median enrichment of WT Salmonella was decreased in the large intestine in germ-free mice compared to SPF mice. Thus, these data imply that PflB-dependent Salmonella colonization is influenced by microbiota-dependent and independent factors. Alternatively, the lack of microbial competitors within germ-free mice may alter the metabolic niche utilized by Salmonella during infection. This notion is supported by our findings that the pflB mutant colonizes at equal levels as a WT strain during single strain infections (FIG. 7G), suggesting that PflB is most critical for bacterial competition and fitness within the intestinal niche in this setting. Furthermore, these data suggest a dependence on host-derived components, particularly within the ileum, during infection. We next assessed whether Salmonella virulence influenced PflB-driven fitness. To do this, we constructed a strain defective for the T3SS encoded within SPI-1 (ΔinvA) and SPI-2 (ΔssaV). This avirulent ‘ΔSPI-1ΔSPI-2’ strain was competed against a corresponding pflB mutant (ΔSPI-1ΔSPI-2ΔpflB). Interestingly, the competitive index was significantly reduced in the absence of Salmonella virulence within the ileum, but not the large intestine (FIG. 7H). Elegant research has focused on the type of cell death, the subsequent pro-inflammatory response generated against Salmonella infection, and the ways in which this inflammatory response can promote intestinal infection^{36,39-42}. Salmonella can induce SPI-1 and SPI-2 dependent regulated cell death, including apoptosis and caspase-1 and 11-dependent pyroptosis, in mammalian cells⁴³⁻⁴⁵. When we assessed the levels of apoptosis in the ileum and colons of infected mice, we observed increased levels of cleaved caspase-3 in the ilea of WT infected mice (data not shown). In contrast, cleaved caspase-3 was undetectable within the colons of infected mice (data not shown). Importantly, ileal cleaved caspase-3 was dependent on the SPI-1/SPI-2 virulence mechanisms (data not shown). PflB-dependent growth and expression was independent of the pyroptotic caspase-1 and caspase-11 in vitro and in vivo (FIG. 7I-J). While Vil-Cre^{+/-} Caspase-3/71^{fl/fl} mice display similar levels of morbidity and bacterial burden compared to littermate controls (FIG. 7K-L, data not shown), the ability of WT Salmonella to outcompete the ΔpflB mutant strain was completely ablated in the ilea of Vil-Cre^{+/-} Caspase-3/7^{fl/fl} mice, with the competitive index returning to 1 (FIG. 7M-N). Together, our data demonstrate that Salmonella virulence factor-dependent epithelial cell apoptosis creates a tissue-specific niche for PflB-driven fitness. Collectively, these data reveal: (i) the in vivo relevance of the apoptosis-dependent transcriptional response induced in bacteria, and the relevance of the pflB gene in models of foodborne Salmonella infection; and (ii) that PflB-dependent bacterial outgrowth during Salmonella-induced colitis is critical throughout infection and that caspase-3/7 dependent epithelial cell apoptosis influences this key fitness strategy in vivo.

### Example 5. TNF-Associated Mammalian Cell Death Enhances Enterobacteriaceae Growth

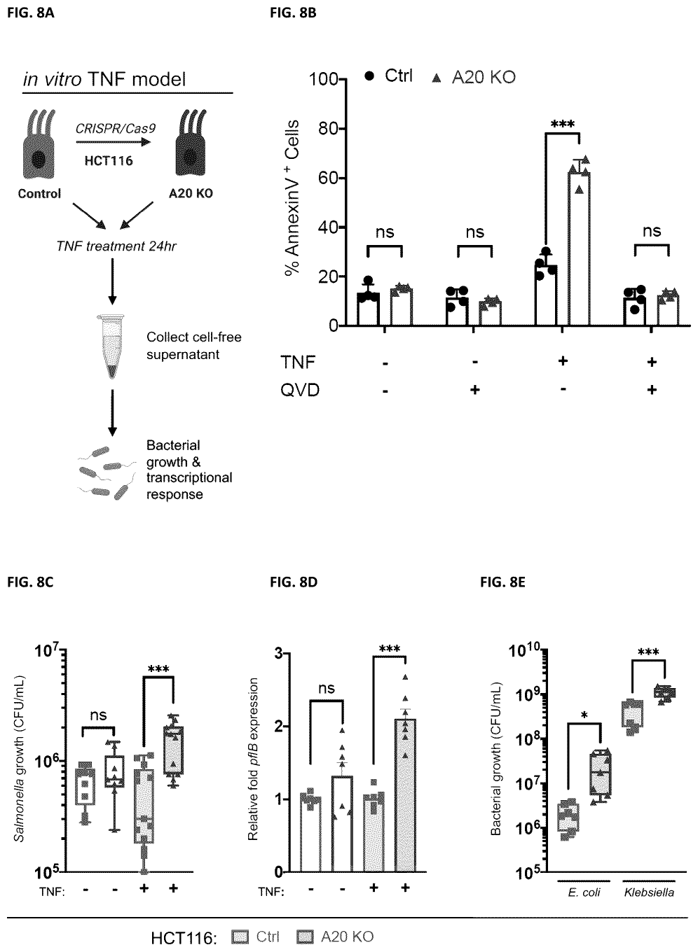

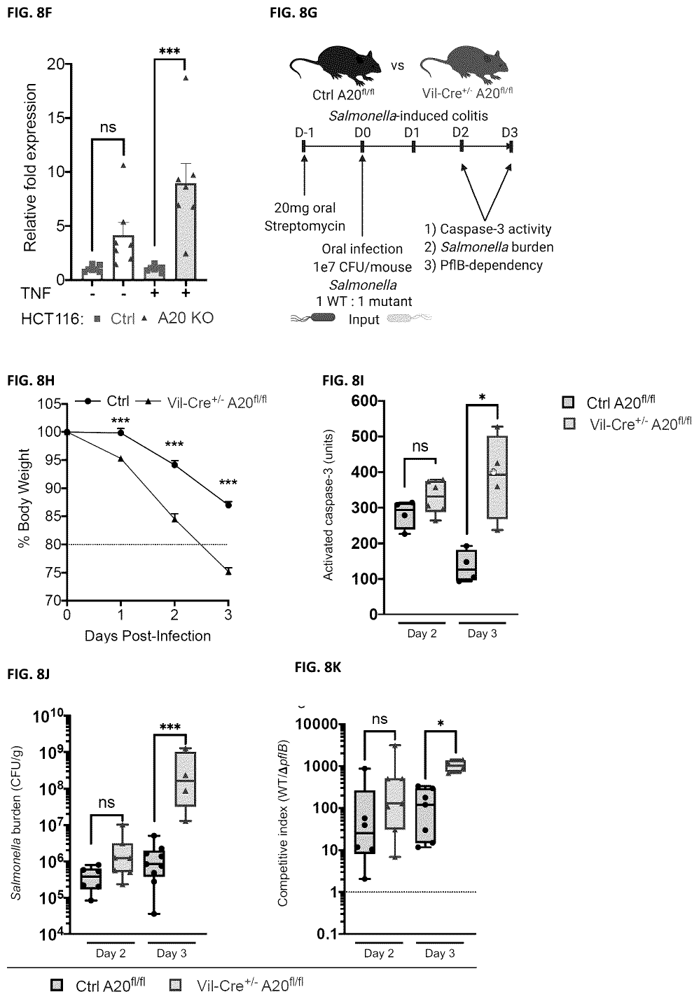

A close association exists between microbial dysbiosis and inflammatory bowel disease (IBD). Patients with flare ups of IBD typically display increased levels of intestinal apoptosis¹⁰, with Enterobacteriaceae outgrowth linked to exacerbated symptoms⁴⁶. TNF is a critical cytokine that promotes intestinal inflammation (e.g. IBD), and anti-TNF therapy can ameliorate the prevalence of intestinal epithelial cell apoptosis in patients⁴⁷. To address whether TNF-induced epithelial cell death might affect Enterobacteriaceae outgrowth, we focused on A20, a known IBD susceptibility gene that functions as a ‘brake’ on TNF induced inflammation⁴⁸. In mouse models, targeted deletion of A20 in gut epithelial cells (in Vil-Cre^{+/-} A20^{fl/fl} mice) results in increased ileal pathology during TNF-induced enteritis, which correlates with increased sensitivity of A20 knockout intestinal epithelial cells to TNF induced apoptosis⁴⁹. As a first step, we generated A20 knockout HCT116 cells and assessed the impact of TNF-induced cell death on bacterial outgrowth (schematically shown in FIG. 8A). TNF treatment induced apoptotic caspase activation, and cell death in A20 knockout cells that was caspase-dependent (FIG. 8B). Supernatants of A20 knockout HCT116 cells treated with TNF significantly promoted Salmonella growth compared to control HCT116 cells treated with TNF (FIG. 8C). Further, this correlated with induction of Salmonella pflB (FIG. 8D) and cadB (FIG. 8F) transcription. Additionally, TNF-induced death promoted similar 5- to 10-fold growth increases of alternative Enterobacteriaceae species (FIG. 8E), suggesting that inflammation-induced apoptosis can drive bacterial outgrowth in vitro.

To assess whether TNF-induced intestinal epithelial cell death enhances bacterial growth in vivo, we chose the model of Salmonella infection (as in FIG. 7A), which induces significant TNF expression and provides an endogenous source of TNF within the intestinal tract⁵⁰ (schematically shown in FIG. 8G). Additionally, Vil-Cre^{+/-} A20^{fl/fl} mice display TNF-driven ileal apoptosis⁴⁹, thereby providing a model system to test whether increased levels of apoptosis would boost Salmonella growth. Vil-Cre^{+/-} A20^{fl/fl} mice lose significantly more weight during the course of Salmonella infection compared to littermate controls (FIG. 8H), requiring euthanasia by day 3. Over the course of infection, ilea of Vil-Cre^{+/-} A20^{fl/fl} mice showed a striking correlation between an increase in caspase-3 activation (FIG. 8I), an increase in total Salmonella burden (FIG. 8J), and an increase in PflB-dependent Salmonella colonization (FIG. 8K). A20 deficiency had additional impact in the large intestine and in systemic dissemination to the spleen (data not shown). These findings provide a new experimental link between the clinically described TNF-induced apoptosis and the expansion of Gram-negative Enterobacteriaceae, and suggest that disease settings associated with substantial intestinal epithelial cell death could leave the host more susceptible to infections.

### Example 6. Chemotherapy-Induced Apoptosis Fuels Enterobacteriaceae Growth in Vivo

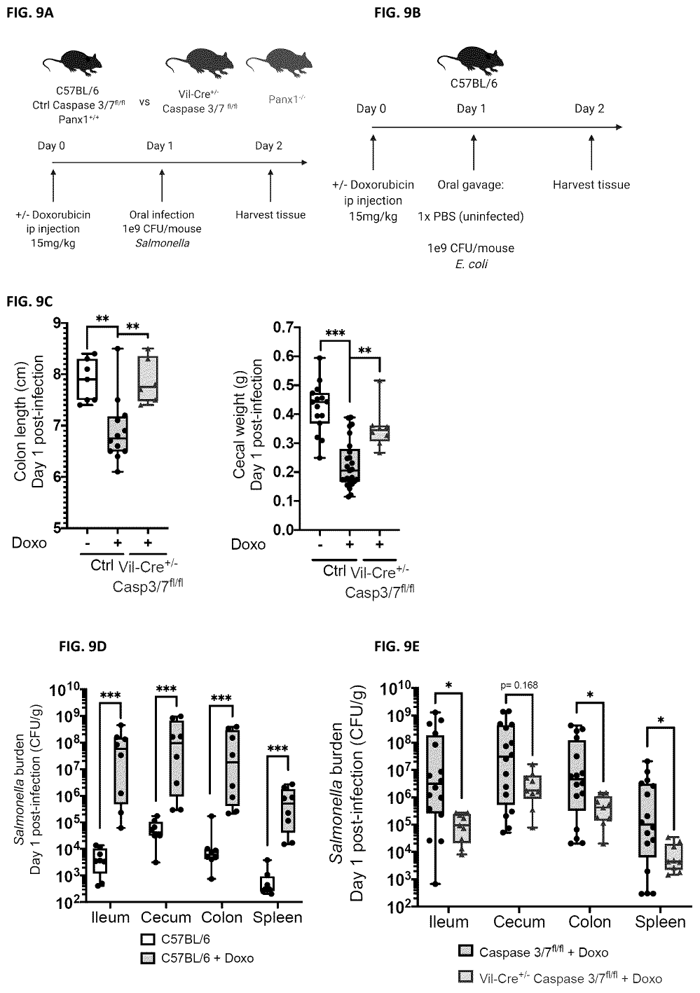

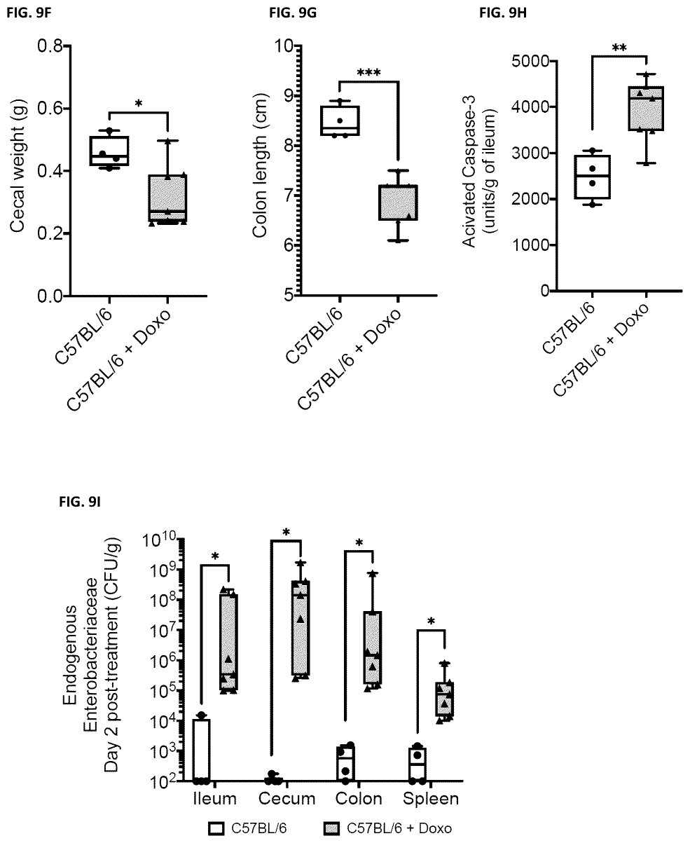

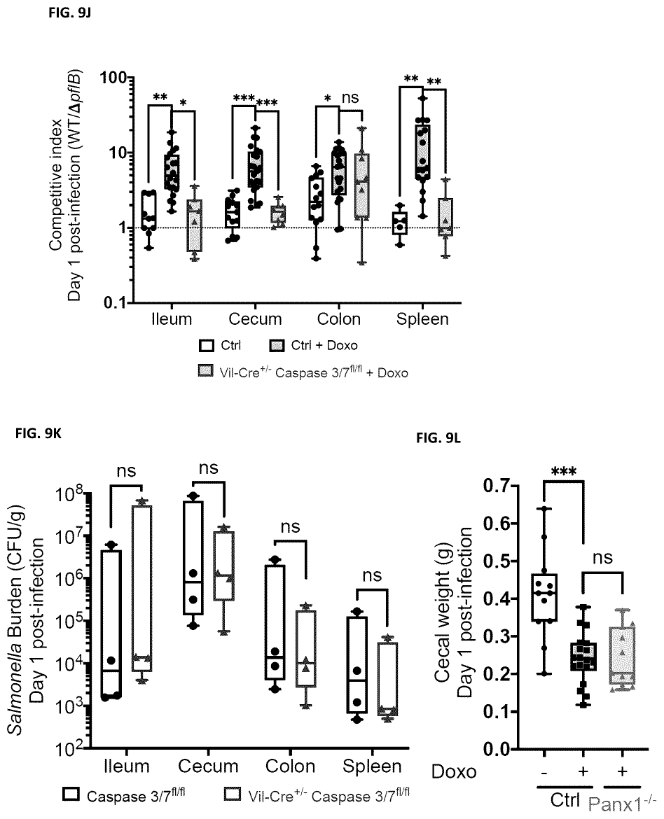

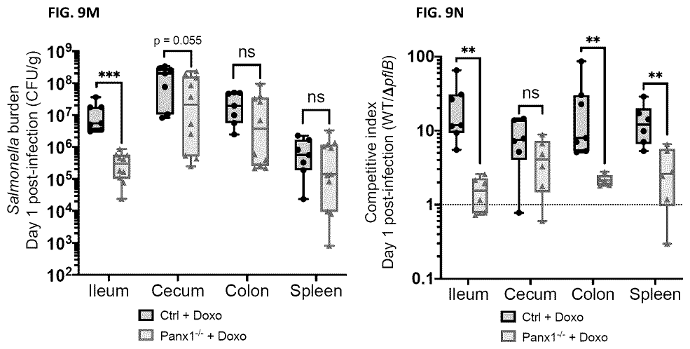

Much like the IBD patient data, cytotoxic chemotherapies are also linked to the enrichment of Proteobacteria in cancer patients⁵¹ and a significantly higher risk of developing infections¹³. While chemotherapy-induced neutropenia can clearly influence susceptibility to infections, the risk of infection is higher in patients with gastrointestinal toxicity and mucositis, independent of the magnitude and duration of neutropenia¹³. These clinical findings suggest additional contributing factors that heighten susceptibility to infection (beyond neutropenia)¹⁴. To test if our findings could contribute to an increase in susceptibility to infection within an in vivo setting of chemotherapy-induced gastrointestinal apoptosis, we utilized a model of doxorubicin-induced gastrointestinal toxicity prior to oral infection with bacteria (schematically shown in FIG. 9A-B). Mice were given a single systemic dose of doxorubicin followed one day later by oral infection with either Salmonella or E. coli. Because there was no streptomycin pretreatment of mice prior to bacterial gavage, this allowed us to address the effects of doxorubicin-induced epithelial apoptosis without the complication of Salmonella-induced colitis³⁴. Doxorubicin treatment induced substantial intestinal pathology, which could be quantified as a significant reduction in cecal weight and a shortening of the colon (data not shown). To directly address the relevance of intestinal epithelial cell apoptosis, we utilized the Vil-Cre^{+/-} Caspase-3/7^{fl/fl} mice. Vil-Cre^{+/-} Caspase-3/7^{fl/fl} mice were significantly protected from doxorubicin-induced intestinal damage compared to control mice (FIG. 9C). In conjunction with intestinal damage, doxorubicin treatment led to staggering increases in bacterial burden following infection with Salmonella (FIG. 9D) or E. coli (data not shown), demonstrating a significant increase in general susceptibility to exogenous Enterobacteriaceae infection in C57BL/6 mice. Given the potential anti-bacterial effects of doxorubicin on the resident microbiota (FIG. 1I), the exogenous Enterobacteriaceae may potently expand to fill a newly unoccupied niche. However, Vil-Cre^{+/-} Caspase-3/7^{fl/fl} mice had significantly reduced levels of Salmonella after doxorubicin treatment compared to littermate controls (FIG. 9E), demonstrating that intestinal epithelial cell apoptosis enhances susceptibility to exogenous Enterobacteriaceae infection in vivo. We next asked whether doxorubicin treatment could contribute to the expansion of endogenous Enterobacteriaceae that is often observed in patients. Doxorubicin treatment alone was sufficient to induce damage to the ceca and colons of uninfected mice (FIG. 9F-G) as well as caspase-3 activation in the ilea (FIG. 9H), indicating that these pathologies were not driven by the exogenous source of infection in this model. In agreement with patient microbiome data, we observed a potent expansion of the endogenous Enterobacteriaceae in uninfected doxorubicin-treated animals, a microbial community that was nearly undetectable in untreated animals (FIG. 9I). Finally, to test the link between doxorubicin-induced apoptosis and the death-dependent growth response of Salmonella, we performed competitive infections with WT and αpflB mutant Salmonella strains and made two key discoveries. First, WT Salmonella was significantly enriched compared to the ΔpflB mutant in control mice after doxorubicin treatment (FIG. 9J). Importantly, WT and the ΔpflB mutant Salmonella were recovered at a nearly 1:1 level in untreated control mice (no doxorubicin, no streptomycin pretreatment) (FIG. 9J), indicating that PflB does not dramatically contribute to Salmonella replication in an otherwise healthy animal. Second, loss of intestinal epithelial apoptosis in Vil-Cre^{+/-} Caspase-3/7^{fl/fl} mice significantly reduced PflB-dependent Salmonella growth after doxorubicin treatment in the ileum, cecum, and spleen (FIG. 9J), but not in the absence of chemotherapy (FIG. 9K). Given the link between Panx1-dependent metabolites and PflB-driven bacterial growth in vitro, we compared control and Panx1^{-/-} mice after doxorubicin treatment. Despite developing similar magnitudes of doxorubicin-induced disease (FIG. 9L), Panx1^{-/-} mice had significantly reduced Salmonella burden and PflB-dependent fitness compared to littermate controls (FIG. 9M-N). These data implicate the metabolites released in response to doxorubicin-induced apoptosis, rather than chemotherapy-induced damage per se, as being critical drivers in the susceptibility to infection. Our findings add a new layer of complexity to chemotherapy-induced susceptibility to bacterial infection, wherein intestinal epithelial cell apoptosis and downstream metabolite release induced by doxorubicin can promote the outgrowth of exogenous and endogenous sources of Enterobacteriaceae within the intestinal tract.

### Discussion

Programmed cell death has many crucial functions that help to maintain tissue homeostasis. In particular, dying cells release soluble factors that permit intercellular mammalian communication that influence cell clearance¹, metabolism²⁰, and the immune response^{39,41,42}. The data presented here provide insights that advance a novel concept that the soluble factors involved in apoptosis-dependent intercellular communication are exploited by intestinal bacteria. First, programmed mammalian death-induced nutrient release (DINNR) can directly fuel bacterial growth, a feature conserved across several apoptotic triggers and the six Enterobacteriaceae family isolates tested. Our findings also demonstrate both the necessity and the sufficiency of epithelial cell apoptosis in promoting bacterial expansion. Although this work focuses on gastrointestinal epithelial cells and intestinal Enterobacteriaceae, we show a conceptual conservation across mammalian cell types (i.e. epithelial cells and lymphocytes) that suggest a broader relevance for these concepts. Second, this work mechanistically identified Salmonella pflB as a critical gene for adaptation within the mammalian host in response to epithelial cell apoptosis in models of foodborne infection, TNF-associated inflammation, and chemotherapy-induced susceptibility to infection. Third, unique bacterial transcriptional responses to apoptotic cell death suggest differences between the various forms of regulated mammalian cell death and the subsequent impact they may have on bacterial outgrowth. Moreover, differences in the transcriptional responses between members within the Enterobacteriaceae (e.g. Salmonella versus E. coli) reflect an added layer of specificity within the bacterial response to a given cell death modality. Finally, these data add a new dimension to the intricate relationship between mammalian cell death and microbial complications, i.e., soluble factors released by apoptotic intestinal epithelial cells leave the host primed for subsequent exogenous infection or promote the dysbiotic outgrowth of the endogenous Enterobacteriaceae. These findings also open the conceptual window for developing supportive therapeutics that might help restrain bacterial growth in patients undergoing cytotoxic chemotherapy or during TNF-associated flare ups.

### Materials and Methods

### Reagents

The reagents used for different parts of this work were obtained from the indicated suppliers as follows: Doxycycline (Sigma D-9891). B/B homodimerizer (Clonetech AP20187). QVD (Sigma SML0063). Staurosporine (Abcam ab120056). TUNEL (Sigma 12156792910). CD45 antibody (Abcam ab10558). Caspase 3 activity kit (Sigma APT131, AssayGenie RG BN00018). Caspase 8 activity kit (Sigma APT129). Annexin V-APC (Biolegend 640941). Annexin V-Pac Blue (Biolegene 640917). TO-PRO-3 Iodide (Thermo Fisher T3605), 7AAD (Invitrogen A1310). Yoyo-1 (Thermo Fisher Y3601). Sytox blue (Thermo Fisher S34857). Pac-1 (Selleckchem S2738). Doxorubicin (Sigma D-1515). Proteinase K (GC Biotech BIO-37037). RiboPure RNA Purification Kit (Thermo Fisher AM1925). SensiFast cDNA Synthesis Kit (GC Biotech BIO-650504). Recombinant human TNF (VIB Protein Core). IMP (Sigma 57510). DHAP (Sigma 37442). GMP (Sigma G8377). UDP-Glucose (Abcam ab120384). Spermidine (Sigma S2626). FBP (Sigma F6803). In Situ Cell Death Detection Kit, TUNEL (Sigma 12156792910). Annexin V binding buffer (BD 556454). Shikonin (MedChemExpress HY-N0822). Pyruvate detection kit (Merck MAK071). Formate detection kit (Sigma MAK059). Fructose 1,6-bisphosphate detection kit (Biovision K2036).

### Antibodies for Western Blot

Caspase-1⁵², Caspase-3 (CST #9662), Cleaved Caspase-3 (CST #9664), Caspase-7 (CST #8438), Cleaved Caspase-7 (Abcam #ab255818), Caspase-8 (Abnova #MAB3429), Cleaved Caspase-8 (CST #9429), Caspase-8 (CST #9746), P-MLKL Ser345 (Abcam #ab196436), MLKL (Sigma-Aldrich #MABC604), P-RIPK3 Thr231/232 (CST #91702), Tubulin HRP (Abcam #ab21058), Sheep Anti-Mouse IgG HRP (Cytiva #NA931), Goat Anti-Rat IgG HRP (Cytiva #NA935), Goat Anti-Rabbit IgG HRP (Cayman Chemical #10004301).

### Mammalian Cell Culture

All mammalian cell culture work was performed using fetal bovine serum from Summerlin Scientific. Serum-free conditions, when applicable, are indicated in the figure legends.

CT26 cells (ATCC CRL-2638) were routinely cultured in DMEM (4.5 g glucose/L) supplemented with 10% FBS, 1× sodium pyruvate, and 1× glutamine or RPMI-1640 containing 10% FBS. CT26:FADD clones were generated previously²⁴. HCT116 cells (ATCC CCL-247) were routinely cultured in McCoy's 5A supplemented with 10% FBS, 1× sodium pyruvate, and 1× glutamine. Jurkat cells (ATCC TIB-152) were cultured in RPMI-1640 containing 10% FBS.

A20 deficient HCT116 cells were generated via CRISPR-Cas9 gene targeting. In short, a sgRNA was designed using the CRISPRscan tool (A20-1:GGAGCTTGTCAGTACATGTG), and cloned into the px458 vector (Addgene plasmid #48138). The day before transfection, 2e10⁶ cells were seeded in a 10-cm cell culture dish. The next day, cells were transfected (jetPEI) with 20 μg plasmid according to the manufacturer's instructions. Two days post transfection, GFP positive single cells were sorted in a 96-well plate. Finally, 7 days post transfection, clones were expanded and screened using western blot analysis, to select the desired A20 KO clones.

### Mammalian Cell Death Induction Assays

All mammalian cells were cultured at 5×10⁵ cells per ml. Cells were washed with 1× PBS before induction of cell death in the indicated medium. Jurkat cells were cultured in suspension at 5×10⁵ cells/ml. After induction of cell death, supernatant was collected and spun at 330 rcf for 5 minutes to remove cellular debris. Unless stated otherwise, the resulting supernatant was filtered using a 0.2 μm syringe filter and either used immediately or frozen at −20° C. for later use. Cells were stained with Annexin V (AV) conjugated to APC or Pac Blue and either Sytox blue, Yoyo-1, or 7AAD (DNA binding dyes) for 15 minutes at room temperature in Annexin V binding buffer (BD). Flow cytometry was performed using the Attune NxT (Invitrogen), the FACS Calibur (BD), the LSR Fortessa (BD), or the LSRII (BD). Data were analyzed using FlowJo v.10 software. All independent experiment values shown are the average values of technical duplicates. ‘% Annexin V⁺’ cells include Annexin V⁺DNA dye⁻ and Annexin V⁺DNA dye⁺ cell populations.

FADD-dependent apoptosis: CT26:FADD clones were incubated with 1 μg/ml doxycycline for 16 hours to induce expression of the construct. Doxycycline was washed away before addition of 10 nM B/B Homodimerizer for 5 hours to induce death. For Caspase inhibition studies, cells were incubated with 30 μM QVD for 1 hour prior to B/B administration and QVD was maintained in the media.

UV-induced apoptosis: HCT116 or CT26 cells were exposed to 600mJ cm⁻² UV-C irradiation (Stratalinker). HCT116 or CT26 cells were incubated for 24 hours after UV irradiation. Jurkat cells were exposed to 150 mJ cm⁻² and then incubated for 4 hours after UV irradiation. For media controls, fresh DMEM was exposed to 600 mJ cm⁻² UV or left unexposed.

Staurosporine-induced apoptosis: HCT116 or CT26 cells were incubated with 1 μM or DMSO vehicle control for 24 hours.

PAC-1-induced apoptosis: CT26 cells were incubated with 50 μM or DMSO vehicle control for 24 hours.

TNF-induced apoptosis: HCT116 cells were treated with 100 ng/ml of recombinant human TNF for 24 hours.

Salmonella-induced cell death: HCT116 cells were infected with a ratio of 100 WT Salmonella to 1 HCT116 cell, spun at 300 rcf for 1 minute to maximize bacterial: epithelial cell contact, and infected for 1 hour. After 1 hour, cells were washed twice with 1× PBS before incubation with media containing 100 μg/ml gentamicin for 30 minutes to kill any extracellular bacteria. Media was then replaced with a lower dose of gentamicin (50 μg/ml) for 24 hours before cell death was quantified via flow cytometry.

Freeze-thaw: CT26 cells were submitted to three cycles of freeze-thaw. For each cycle, cells were frozen solid on dry ice and then thawed in a 37° C. water bath. Control samples remained at room temperature. For supernatant collection, cellular debris was removed, and supernatant filtered as described above.

Shikonin: CT26 cells were pretreated with 5 μM Shikonin (or DMSO vehicle control) for 1 hour. Cells were then washed once with PBS before replacing with fresh media containing the indicated trigger of cell death +/−5 μM Shikonin.

### Immunoblotting

CT26 or HCT116 cells were seeded in a 6-well plates at a concentration of 400,000 cells per well. Cell death was induced as described above with or without pretreatment with 30 μM QVD. After the indicated time of cell death induction (2, 5 or 24 hours), cells were collected and lysed directly in sample buffer. Following protein denaturation, SDS-PAGE was performed using 8% or 4-12% gradient Bis-Tris gels (Caspase-1 detection on 12% Tris-Glycine gels). Primary antibodies were used for overnight incubation, followed by one hour incubation with secondary antibody and chemiluminescence detection.

### Collection and Treatment of Supernatants

After induction of cell death with the indicated trigger, cell-free supernatant was collected and spun at 330 rcf for 5 minutes to remove cellular debris. The resulting supernatant was filtered using a 0.2 μm syringe filter. Media controls underwent matching spins and 0.2 μm filtration. Supernatants or media controls were subsequently directly inoculated with bacteria for growth studies, or frozen at −20° C. for later use.

Metabolite detection: Pyruvate, formate, or fructose 1,6-bisphosphate concentrations were determined using the appropriate detection kit per the manufacturer's instructions. For these experiments, mammalian cells were cultured in DMEM without phenol red supplemented with 10% FBS.

While the supernatants of apoptotic cells were often used as such for their effects on bacterial growth, in specific instances, the supernatants were treated as indicated below.

Sequential filtration: Following Stauro-induced apoptosis, CT26 supernatants were collected and filtered (‘>300 kD’). Supernatants were subjected to sequential filtrations with the indicated size Amicon® Ultra (Merck Millipore) filters by spinning at maximum speed for 10-30 minutes according to the manufacturer's instructions. Aliquots were removed in between each filtration step and either used fresh or frozen at −20° C. for subsequent use.

Proteinase K treatment: Supernatants were sequentially filtered to <10 kD as described above. <10 kD supernatants were then treated with 50 μg/ml Proteinase K and incubated at 37° C. for 1 hour. Control samples were left untreated at 37° C. Following treatment, Proteinase K was removed by subsequent filtration <3 kD. Supernatants were used immediately or frozen at −20° C. for subsequent use.

Protein quantification: Total protein in media, live cell, or apoptotic supernatants was determined using the Pierce™ BCA Protein Assay Kit (Thermo) according to the manufacturer's instructions. Protein concentration (μg/ml) was calculated from albumin standard dilutions. Concentrations were determined +/−FBS in the media, +/−3 kD filtration (as described above), or +/−Proteinase K treatment (as described above).

Temperature denaturation: 0.2 μm-filtered supernatants were left at room temperature or incubated at 100° C. for 15 minutes. Following incubation, supernatants were cooled to room temperature and used immediately or frozen at −20° C. for subsequent use.

### Primary Colonocyte Extraction and Killing

Full length colons (from cecum to rectum) were excised from the indicated genotype of mice and cut open longitudinally to flay the colon open. Opened colons were vortexed 2-3 times in sterile 1× PBS to remove fecal content. Colons were then cut horizontally into 5-7 mm long pieces and incubated in DMEM +10% FBS and an antibiotic cocktail containing Ampicillin (100 μg/ml), Chloramphenicol (20 μg/ml), Kanamycin (50 μg/ml), and Streptomycin (100 μg/ml) for 2 hours at 37° C. with 5% CO₂. Colonic pieces were then washed 3 times in sterile 1× PBS to remove any traces of the antibiotic cocktail. Individual colonic pieces were then incubated in 1.5 ml Eppendorf tubes containing 500 μl of DMEM+10% FBS+/−2 μM Stauro for 8 hours or +/−20 μg/ml Doxorubicin for 6 hours. For caspase inhibition, 30 μM QVD was added to the 2-hour antibiotic treatment step and was maintained throughout death induction. After death induction, colonic explants were spun at max speed for 5 minutes using a tabletop centrifuge and supernatant was collected. Unfiltered supernatant was used immediately or frozen −20° C. for subsequent use. Each colon typically yielded 9-10 individual explants. All subsequent experiments (cell death, bacterial growth) utilized explants extracted from a minimum of 3 mice.

Caspase-3 activation: Following removal of the supernatant, explant pieces were assessed for caspase-3 activity via the Caspase-3 Colorimetric Activity Assay Kit, DEVD (Sigma) according to the manufacturer's instructions. Colorimetric values were obtained at 405 nm using the iMark™ microplate reader (BioRad) and arbitrary units were calculated from a standard curve using the provided pNA standard.

Microscopy: Colonic explant samples were fixed overnight in 4% paraformaldehyde, embedded in paraffin, and cut at 5 μm thickness. Subsequently, sections were stained with hematoxylin and eosin or a TUNEL/CD45/DAPI co-staining. The TUNEL assay was done according to manufacturer's instructions (In situ cell death detection kit, TMR red-Roche) and followed by an overnight incubation at 4° C. with DAPI and a rabbit CD45 (Abcam ab10558) antibody. CD45 was visualized with an anti-rabbit secondary antibody coupled to Dylight633.

For cleaved caspase 3 staining, tissues were fixed overnight in 4% neutral buffered formalin, embedded in paraffin and cut into 5-μm sections. Slides were deparaffinized, rehydrated with an alcohol series and processed for antigen retrieval with Dako citrate buffer (Agilent, S169984-2) for 25 min in a pressure cooker. Endogenous peroxidase activity was blocked using Dako REAL Peroxidase-Blocking Solution for 20 min (Agilent S202386-2), followed by 3 washes in PBS (5 min each). Sections were blocked for 30 min in Dako REAL Antibody Diluent (Agilent, S202230-2) supplemented with 5% goat serum (Agilent, X090710-8). Incubation with cleaved caspase-3 antibody (Cell Signaling Technology, 9664S, 1:1,500) was carried out overnight at 4° C. in blocking buffer. Sections were washed 3 times with PBS (5 min each) and incubated with SignalStain Boost IHC Detection Reagent (Cell Signaling Technology, 8114S) for 30 min. Signal was developed using ImmPACT DAB Substrate (Vector Laboratories, SK-4105) followed by hematoxylin counterstaining, dehydration and mounting with Entellan new (Merck Millipore, 107906).

Slides were imaged with an Axio Scan.Z1 (Zeiss, Jena, Germany), using a 10× Plan-Apochromat 0.45 NA (0.650 μm/pixel) and a Hamamatsu Orca Flash camera. With an HXP illumination source, the following were used in the acquisition: DAPI (BP 445/50), HE GFP (BP 525/50), HE DsRed (BP 605/70), and Cy5 (BP 690/50). Image analysis was performed using QuPath (version 0.1.2).

### Bacterial Growth Experiments

Overnight cultures of the indicated organism were routinely grown at 37° C. with 200 rpm agitation (aerobic) in LB broth. Following overnight culture (approximately 16 hours), 1 ml of bacterial culture was pelleted and media removed. Cultures were re-suspended in 1× PBS.

OD₆₀₀ measurements: 4 ml of supernatant or media controls were inoculated with 1-2×10⁷ CFU/ml of the indicated bacterial species. Cultures were grown at 37° C. with 200 rpm agitation (aerobic) and bacterial growth was quantified by OD₆₀₀ measurements using the Ultrospec 10 (VWR) at the indicated time points. Growth of gentamicin-resistant Salmonella in infected supernatants used a starting inoculum of 8×10⁷ CFU/ml. All independent experiment values are the average values of technical duplicates.

CFU/ml values: 500 μl of supernatant or media controls were inoculated with 1×10³ CFU of the indicated strain. Cultures were grown at 37° C. with 200 rpm agitation (aerobic) and growth was assessed at the indicated time by serially diluting with 1× PBS and plating onto LB agar (in vitro supernatants) or MacConkey agar (ex vivo primary colonocyte supernatants) to obtain CFU/ml values.

‘MeMix6’ supplementation: The metabolite mixture ‘MeMix 6’ was composed of spermidine, FBP, DHAP, GMP, IMP, and UDP-Glucose¹⁹. A single mixture containing the indicated concentrations (FIG. 6B) of each metabolite was prepared fresh in 1× PBS and diluted to the indicated final concentrations in DMEM with 10% FBS. Indicated concentrations of each metabolite were based on previously published targeted metabolomic studies¹⁹.

### Bacterial Mutagenesis

Salmonella mutant strains are listed in Table 2 and were constructed using lambda red homologous recombination as previously described using the LR primers and plasmids listed in Table 2 and Table 3⁵³. Correct polar insertions, antibiotic resistance profiles, and subsequent nonpolar deletions following pCP20 transformation and flippase activity were verified using primers listed in Table 1. A similar approach was taken to generate gentamicin resistant Salmonella with slight modifications. Instead of helper plasmid pKD4, the gentamicin resistant gene was amplified from plasmid pRGD (Addgene #74106) with flanking regions of homology to the downstream region of the essential gene glmS. Chromosomal insertion here has been shown to ensure sufficient expression⁵⁴. The pflB mutant was complemented with plasmid pLK003. Plasmid based complementation was achieved using the arabinose-inducible pBAD24 vector, amplification of Salmonella genomic DNA using the primers listed in Table 1, and EcoR1 and HindIII restriction enzymes. As controls, WT and ΔpflB strains were transformed with empty pBAD24 vector.

### Bacterial Gene Expression

Primer sequences are listed in Table 1. For all gene expression studies, bacterial cultures were grown to mid logarithmic growth phase (OD₆₀₀=0.4−0.6), spun down at max speed, and then resuspended in TRIzol™ reagent (ThermoFisher) for immediate extraction or frozen at −80° C. Bacterial RNA was extracted and DNA removed as described using the RiboPure RNA Purification Kit (Thermo Fisher). For RNAseq experiments, ribosomal RNA was removed using Ribo-zero kits (Illumina). For all RNAseq data, sample groups were analyzed in quadruplicates and reads were mapped to the Salmonella Typhimurium SL1344 reference genome (ASM21085v2, EnsEMBL 39).

RNA sequencing and analysis: For the HCT116+/−UV data, production and analysis were performed by the VIB Nucleomics Core (www.nucleomics.be) on the Illumina NextSeq platform. An mRNA library was constructed using the TruSeq Stranded mRNA kit (Illumina), sequencing was conducted using the NextSeq High Output, and differential gene expression and statistical significance was determined by edgeR (Bioconductor). The CT26:FADD data were produced and analyzed by Novogene. An mRNA library was constructed following rRNA depletion using NEBNext Ultra RNA Library Prep Kit for Illumni (NEB), sequencing was performed on the Illumina PE150 platform, and differential gene expression and significance was determined using HTSeq software.

qPCR: cDNA synthesis was performed using the SensiFast cDNA synthesis kit (GC Biotech). qPCR primers were designed using NCBI Primer Blast. The ΔΔCT values for each independent biological replicate were calculated twice, first using the Salmonella housekeeping gene gmk⁵⁵ and then the housekeeping gene strB⁵⁶ and the two ΔΔCT values were averaged. Relative fold expression was calculated such that the average of the indicated controls was equal to 1. The endogenous controls for HS E. coli samples were gmk and rpoA⁵⁷. All independent experiment values shown are the average values of technical duplicates.

### Animal Studies

All animal work was approved by the University of Virginia, the VIB-UGent Center for Inflammation Research, and the University of Ghent ethical committees. WT C57BL/6 mice were purchased from Janvier Labs. Global caspase-1/11 KO were a gift from Mo Lamkanfi. Vil-Cre^{+/-} Caspase-3/7^{fl/fl}, Vil-Cre^{+/-} A20^{fl/fl}, RIPK1^{KD}, MLKO^{-/-}, GasderminD^{-/-}, and Panx1^{-/-} mice have been described previously and were housed at the VIB-UGent Center for Inflammation Research^{21,49,58-60}. For all mutant mouse genotypes, fl/fl Cre-negative mice served as littermate controls where appropriate. Vil-Cre^{+/-} mice were co-housed with Vil-Cre^{-/-} controls during development and genotypes were only separated at the beginning of each experiment. Panx1^{-/-} mice were similarly co-housed with control Panx1^{+/+} mice until experimentation. Germ-free experiments were done in the germ-free facility at the University of Ghent. All mice were 7-14 weeks of age at the time of infection, and both male and female mice were used as indicated.

Salmonella-induced colitis. Littermate, sex, and age-matched mice were infected using the Salmonella model of colitis³⁴. Specific pathogen free (SPF) mice were given a single dose of 20 mg streptomycin via oral gavage (100 μl of 200 mg/ml streptomycin dissolved in water) one day prior to infection. Mice were infected with 1×10⁷ CFU per mouse resuspended in 1× PBS via oral gavage. For competitive infections, polar deletions containing the chromosomally inserted kanamycin resistance gene were used⁵⁶. Mice were infected with 1×10⁷ CFU per strain per mouse, for a 2×10⁷ CFU per mouse total inoculum in 100 μl. Germ-free mice were infected with 2×10⁶ CFU per mouse (1×10⁶ CFU per strain) and did not receive streptomycin. The input ratio of each strain was calculated by plating the infective dose on agar plates containing streptomycin (total inoculum) and agar plates containing streptomycin plus kanamycin (mutant strain). WT Salmonella was thus calculated as (total inoculum)−(mutant inoculum) and input ratio was calculated as (WT Salmonella)/(mutant Salmonella inoculum) with a desired input equal to 1. Mouse body weight was measured daily and ‘% body weight’ was calculated as (daily weight)/(day 0 starting weight). At the indicated day post-infection, intestinal tissue (ileum, cecum, colon) and the spleen were harvested. For bacterial burden measurements and cleaved caspase-3 staining, luminal contents of the ileum (the last 5-6 cm of the distal end of the small intestine) and the colon were removed. Luminal content was retained in all cecal samples. Any attaching lymphatic tissue was removed from intestinal tissue before homogenization in 1 ml of 1× PBS. CFU per gram of tissue were calculated by plating serial dilutions of tissue homogenates on MacConkey agar containing streptomycin and MacConkey agar containing streptomycin and kanamycin. Single strain infection tissue homogenates were plated solely on MacConkey agar containing streptomycin.

Germ-free mice. Axenic/germ-free mice were housed in positive-pressure flexible film isolators (North Kent Plastics). One week before the start of the infection experiment, axenic mice were transferred to individually ventilated Isocage-P cages (positive pressure Isocages-Techniplast). All experiments were performed on mice of C57BL/6J genetic background. All experiments on axenic mice were performed according to institutional (ethical committee for animal experimentation Ghent University-Faculty Medicine and Health Sciences), national and European animal regulations.

Doxorubicin treatment. Doxorubicin was given as a single intraperitoneal (ip) injection at 15 mg/kg of mouse body weight while vehicle control (water) was given at similar volumes (approximately 300 μl volumes of water or 1 mg/ml Doxorubicin solution). The next day, mice were infected with 1×10⁹ CFU of Salmonella (either single strain of competitive infections, as described above) or 1×10⁹ CFU of E. coli. Tissues were harvested at day 1 post-infection and weight loss, total Salmonella burden, and competitive indices were calculated as above. Total Enterobacteriaceae burden, including E. coli, was assessed by plating tissue lysates on MacConkey agar without antibiotics. Uninfected controls were given 1× PBS via oral gavage instead of bacteria, and Endogenous Enterobacteriaceae burden was assessed by plating tissue lysates on MacConkey agar without antibiotics.

### Statistical Analysis

All box and whiskers plots show minimum to maximum with all independent replicates included, and the horizontal line within the box depicting the median. Statistical tests were performed using GraphPad Prism version 8 as indicated in the figure legends. Outlier data points were identified using the ROUT method with Q=1%.

### Tables

TABLE 1
Primer sequences

| Primer | Sequence | Use |
|---|---|---|
| cadBA_LR_F1 | 5′-ATGAACATGAGTTCTGTCAAAAAGATCGGGCT | Lambda-red |
| | ATTTGCCTGTACTGGCGTGTGTAGGCTGGAGCT | mutagenesis |
| | GCTTC-3′ | |
| cadBA_LR_R1 | 5′-GTACTTATTTCGTATTTTCTTTCAGCACCT | Lambda-red |
| | TAACGGTGTAACGTCCGTCTCATATGAATATCC | mutagenesis |
| | TCCTTAG-3′ | |
| cadBA_F1 | 5′-TAGGACTTATCTCAACCCATTCGT-3′ | PCR check |
| cadBA R1 | 5′-TTGATCGCGTGGCTGTCAT-3′ | PCR check |
| pflB_LR_F1 | 5′-GTGTTACATGTCCGAGCTTAATGAAAA | Lambda-red |
| | GTTAGCCACAGCCTGGGAAGGTTGTGTAGG | mutagenesis |
| | CTGGAGCTGCTTC-3′ | |
| pflB_LR_R1 | 5′-CCCATTACATGGTCTGCGTGAAGGTACGAG | Lambda-red |
| | TAATAACGTCCTGCTGCTGTCATATGAATATCC | mutagenesis |
| | TCCTTAG-3′ | |
| pflB_F1 | 5′-CTCACCTGACCGTGATGAGC-3′ | PCR check |
| pflB_R1 | 5′-GTGAAAAGTCGAGTTGTCGCAAT-3′ | PCR check |
| pKD4_F1 | 5′-CCTTCTATGAAAGGTTGGGC-3′ | PCR check |
| pKD4_R1 | 5′-TGTCCAGATAGCCCAGTAG-3′ | PCR check |
| gentR_LR_F1 | 5′-AATGTAGTACCAGGCAGAAGGCCGTCGACAGACGG | Lambda-red |
| | CCTTTTTTTGTGCGCATGTTTGATGTTATGGAGCA-3′ | mutagenesis |
| gentR_LR_R1 | 5′-CTGGATGCGACTCAAAAGCCCCGACCGGGCCAGG | Lambda-red |
| | GGGATCCACCTGAATAAAGCCGATCTCGGCTTGAAC-3′ | mutagenesis |
| pRGD_gentR_F1 | 5′-ATGTTTGATGTTATGGAGCA-3′ | PCR check |
| pRGD_gentR_R1 | 5′-AAGCCGATCTCGGCTTGAAC-3′ | PCR check |
| glmS_insert check | 5′-CTTACATTCACGCGGAAGCC-3′ | PCR check |
| invA_LR_F1 | 5′-CCTATAGTGCTGCTTTCTCTACTTAACAGTG | Lambda-red |
| | CTCGTTTACGACCTGAATTGTGTAGGCTGGAGCT | mutagenesis |
| | GCTTC-3′ | |
| invA_LR_R1 | 5′-GCCCTTATATTGTTTTTATAACATTCACTGACT | Lambda-red |
| | TGCTATCTGCTATCTCACATATGAATATCCTCCTT | mutagenesis |
| | AG-3′ | |
| invA_F1 | 5′-AATGGCAGAACAGCGTCGTA-3′ | PCR check |
| invA_R1 | 5′-GCGCCCAGATCCATACATCA-3′ | PCR check |
| ssaV_LR_F1 | 5′-gtcgcaATGCGTTCATGGTTAGGTGA | Lambda-red |
| | GGGAGTCAGGGCGCAACAGTGGCTGTGT | mutagenesis |
| | AGGCTGGAGCTGCTTC-3′ | |
| ssaV_LR_R1 | 5′-CAATTCATTCTTCATTGTCCGCCAACTCCTCT | Lambda-red |
| | TCGCTAAGGTCAATACTTCATATGAATATCCT | mutagenesis |
| | CCTTAG-3′ | |
| ssaV_F1 | 5′-ACTGCTTTTTAAAGCGTTGGCT-3′ | PCR check |
| ssaV_R1 | 5′-ACAGCACAACTCGCCCATAA-3′ | PCR check |
| pflB_pBAD24 | 5′-CTAGGAATTCATGTCCGAGCTTAATGAAAA-3′ | Complementation |
| comp_F2 | | |
| pflB_pBAD24 | 5′-CTAGAAGCTTTTACATGGTCTGCGTG | Complementation |
| comp_R1 | AAGGTACGAG-3′ | |
| gmk_qPCR_F1 | 5′-AACCATGATTGGCAGGGAGG-3′ | qPCR |
| | | housekeeping |
| gmk_qPCR_R1 | 5′-GTTGCCAGCACTTGCTCAAT-3′ | qPCR |
| | | housekeeping |
| strB_qPCR_F1 | 5′-CTACCAGGCGACCGAAATTG-3′ | qPCR |
| | | housekeeping |
| strB_qPCR_R1 | 5′-GTTCCTCAGATGCGGCATACA-3′ | qPCR |
| | | housekeeping |
| cadB_qPCR_F1 | 5′-TGAAAAACCCGAAACGCACC-3′ | qPCR |
| cadB_qPCR_R1 | 5′-GAACATCCCGGCGATAACCT-3′ | qPCR |
| pflB_qPCR_F1 | 5′-ATGCGTCCGGACTTCAACAA-3′ | qPCR |
| pflB_qPCR_R1 | 5′-ATGGTTTTCGCCAGGTTTGC-3′ | qPCR |
| E. coli_ | 5′-AAAGGCGAGGATCTGGAAGC-3′ | qPCR |
| pflB_qPCR_F1 | | |
| E. coli_ | 5′-GCCGGGCGAGAGATATCAAA-3′ | qPCR |
| pflB_qPCR_R1 | | |
| E. coli_ | 5′-TTCGCCGCTAAAGTGTCCAT-3′ | qPCR |
| pflB2_qPCR_F1 | | |
| E. coli_ | 5′-CGCAGCAAGCAATAGCGTAG-3′ | qPCR |
| pflB2_qPCR_R1 | | |
| E. coli_ | 5′-ATATGCTCGGCGGTACTTGG-3′ | qPCR |
| cadB_qPCR_F1 | | |
| E. coli_ | 5′-CCGCATCAAACCAATGCCAG-3′ | qPCR |
| cadB_qPCR_R1 | | |
| E. coli_ | 5′-CGCGGTCGTGGTTATGTG-3′ | qPCR |
| rpoA_qPCR_F1 | | housekeeping |
| E. coli_ | 5′-GCGCTCATCTTCTTCCGAAT-3′ | qPCR |
| rpoA_qPCR_R1 | | housekeeping |
| E. coli_ | 5′-GCAGCAAATTCGCCAGAAGA-3′ | qPCR |
| gmk_qPCR_F1 | | housekeeping |
| E. coli_ | 5′-TGCAATGACCTCTTCGCTGT-3′ | qPCR |
| gmk_qPCR_R1 | | housekeeping |

TABLE 2
Bacterial Strains

| Strain | Description |
|---|---|
| Salmonella enterica | WT Salmonella strain SL1344⁶¹ |
| serovar | |
| Typhimurium | |
| E. coli | Commensal E. coli strain HS⁶² |
| Klebsiella | ATCC 43816 KPPR1⁶³ |
| pneumoniae | |
| Adherent Invasive | LF82, isolated from Crohn's Disease patient²⁷ |
| E. coli (AIEC) | |
| E. coli | CCR20, isolated from colorectal cancer patient²⁸ |
| Uropathogenic | UTI189, isolated from urinary tract infection |
| E. coli (UPEC) | patient²⁹ |
| CJA001 | SL1344 gentamicin resistant |
| CJA033 | SL1344 ΔcadBA polar mutant, kanamycin resistant |
| CJA042 | SL1344 ΔcadBA nonpolar mutant |
| CJA057 | SL1344 ΔpflB polar mutant, kanamycin resistant |
| CJA071 | SL1344 ΔpflB nonpolar mutant |
| CJA077 | SL1344 ΔinvAΔssaV (‘ΔSPI-1ΔSPI-2’) nonpolar |
| | mutant |
| CJA081 | SL1344 ΔinvAΔssaVΔpflB (‘ΔSPI-1ΔSPI-2ΔpflB’) |
| | polar mutant, kanamycin resistant |
| LK010 | SL1344 + pBAD 24, empty vector control |
| LK037 | SL1344 ΔpflB (CJA071) + pBAD 24, empty vector |
| | control |
| LK049 | SL1344 ΔpflB (CJA071) + pLK003, pflB insert into |
| | pBAD24 vector |

TABLE 3
Plasmids

| Plasmid | Description | Use | Reference |
|---|---|---|---|
| pKD46 | Lambda-red | mutagenesis | 53 |
| | recombinase | | |
| pKD4 | Kanamycin resistance | mutagenesis | 53 |
| | helper plasmid | | |
| pCP20 | Flippase | mutagenesis | 53 |
| pRGD-GMR | Gentamicin resistance | mutagenesis | 64 |
| pBAD24 | Arabinose inducible | complement | 65 |
| pLK003 | pflB insert into | complement | this study |
| | pBAD24 | | |

### REFERENCES

1. Boada-Romero, E., Martinez, J., Heckmann, B. L. & Green, D. R. The clearance of dead cells by efferocytosis. Nature Reviews Molecular Cell Biology (2020) doi: 10.1038/s41580-020-0232-1.
2. Doran, A. C., Yurdagul, A. & Tabas, I. Efferocytosis in health and disease. Nature Reviews Immunology (2020) doi: 10.1038/s41577-019-0240-6.
3. Ke, F. F. S. et al. Embryogenesis and Adult Life in the Absence of Intrinsic Apoptosis Effectors BAX, BAK, and BOK. Cell (2018) doi: 10.1016/j.cell.2018.04.036.
4. Vince, J. E. & Silke, J. The intersection of cell death and inflammasome activation. Cellular and Molecular Life Sciences (2016) doi: 10.1007/s00018-016-2205-2.
5. Schwarzer, R., Jiao, H., Wachsmuth, L., Tresch, A. & Pasparakis, M. FADD and Caspase-8 Regulate Gut Homeostasis and Inflammation by Controlling MLKL- and GSDMD-Mediated Death of Intestinal Epithelial Cells. Immunity (2020) doi: 10.1016/j.immuni.2020.04.002.
6. Honda, K. & Littman, D. R. The microbiota in adaptive immune homeostasis and disease. Nature (2016) doi:10.1038/nature18848.
7. Bäumler, A. J. & Sperandio, V. Interactions Between the Microbiota and Pathogenic Bacteria in the Gut. Nature 535, 85-93 (2016).
8. Charbonneau, M. R. et al. A microbial perspective of human developmental biology. Nature (2016) doi:10.1038/nature18845.
9. Storek, K. M. & Monack, D. M. Bacterial Recognition Pathways That Lead to Inflammasome Activation. Immunol. Rev. 265, 112-129 (2015).
10. Iwamoto, M., Koji, T., Makiyama, K., Kobayashi, N. & Nakane, P. K. Apoptosis of crypt epithelial cells in ulcerative colitis. J. Pathol. (1996) doi:10.1002/(SICI) 1096-9896 (199610)180:2<152:AID-PATH649>3.0.CO; 2-Y.
11. Lane, E. R., Zisman, T. L. & Suskind, D. L. The microbiota in inflammatory bowel disease: Current and therapeutic insights. Journal of Inflammation Research (2017) doi:10.2147/JIR.S116088.
12. Boussios, S., Pentheroudakis, G., Katsanos, K. & Pavlidis, N. Systemic treatment-induced gastrointestinal toxicity: Incidence, clinical presentation and management. Annals of Gastroenterology (2012).
13. Elting, L. S. et al. The burdens of cancer therapy: Clinical and economic outcomes of chemotherapy-induced mucositis. Cancer (2003) doi:10.1002/cncr.11671.
14. Nesher, L. & Rolston, K. V. I. The current spectrum of infection in cancer patients with chemotherapy related neutropenia. Infection (2014) doi:10.1007/s15010-013-0525-9.
15. Shin, N. R., Whon, T. W. & Bae, J. W. Proteobacteria: Microbial signature of dysbiosis in gut microbiota. Trends in Biotechnology (2015) doi:10.1016/j.tibtech.2015.06.011.
16. Tanzer, M. C. et al. Quantitative and Dynamic Catalogs of Proteins Released during Apoptotic and Necroptotic Cell Death. Cell Rep. (2020) doi:10.1016/j.celrep.2019.12.079.
17. Lauber, K. et al. Apoptotic cells induce migration of phagocytes via caspase-3-mediated release of a lipid attraction signal. Cell (2003) doi:10.1016/S0092-8674(03)00422-7.
18. Elliott, M. R. et al. Nucleotides Released by Apoptotic Cells Act as a Find-Me Signal to Promote Phagocytic Clearance. Nature 461, 282-286 (2009).
19. Medina, C. B. et al. Metabolites released from apoptotic cells act as tissue messengers. Nature (2020) doi:10.1038/s41586-020-2121-3.
20. Yurdagul, A. et al. Macrophage Metabolism of Apoptotic Cell-Derived Arginine Promotes Continual Efferocytosis and Resolution of Injury. Cell Metab. (2020) doi:10.1016/j.cmet.2020.01.001.
21. Saavedra, P. H. V. et al. Apoptosis of intestinal epithelial cells restricts Clostridium difficile infection in a model of pseudomembranous colitis. Nat. Commun. (2018) doi:10.1038/s41467-018-07386-5.
22. Christgen, S. et al. Identification of the PANoptosome: A Molecular Platform Triggering Pyroptosis, Apoptosis, and Necroptosis (PANoptosis). Front. Cell. Infect. Microbiol. 10, 237 (2020).
23. Wang, Y. et al. Chemotherapy drugs induce pyroptosis through caspase-3 cleavage of a gasdermin. Nature 547, 99-103 (2017).
24. Aaes, T. L. et al. Immunodominant AH1 Antigen-Deficient Necroptotic, but Not Apoptotic, Murine Cancer Cells Induce Antitumor Protection. J. Immunol. (2020) doi:10.4049/jimmunol.1900072.
25. Rasko, D. A. et al. The Pangenome Structure of Escherichia coli: Comparative Genomic Analysis of E. coli Commensal and Pathogenic Isolates. J. Bacteriol. 190, 6881-6893 (2008).
26. Hayden, M. K. et al. Prevention of colonization and infection by klebsiella pneumoniae carbapenemase-producing enterobacteriaceae in long-term acute-care hospitals. Clin. Infect. Dis. (2015) doi:10.1093/cid/ciu1173.
27. Darfeuille-Michaud, A. et al. Presence of adherent Escherichia coli strains in ileal mucosa of patients with Crohn's disease. Gastroenterology 115, 1405-1413 (1998).
28. Cougnoux, A. et al. Bacterial genotoxin colibactin promotes colon tumour growth by inducing a senescence-associated secretory phenotype. Gut 63, 1932-1942 (2014).
29. Spaulding, C. N. et al. Selective depletion of uropathogenic E. coli from the gut by a FimH antagonist. Nature 546, 528-532 (2017).
30. Dell, C. L., Neely, M. N. & Olson, E. R. Altered pH lysine signalling mutants of cadC, a gene encoding a membrane-bound transcriptional activator of the Escherichia coli cadBA operon. Mol. Microbiol. (1994) doi:10.1111/j.1365-2958.1994.tb01262.x.
31. Knappe, J. & Sawers, G. A radical-chemical route to acetyl-CoA: the anaerobically induced pyruvate formate-lyase system of Escherichia coli. FEMS Microbiol. Lett. (1990) doi:10.1016/0378-1097(90)90689-N.
32. Chen, J. et al. Shikonin and its analogs inhibit cancer cell glycolysis by targeting tumor pyruvate kinase-M2. Oncogene 30, 4297-4306 (2011).
33. Chekeni, F. B. et al. Pannexin 1 Channels Mediate ‘find-Me’ Signal Release and Membrane Permeability During Apoptosis. Nature 467, 863-867 (2010).
34. Barthel, M. et al. Pretreatment of Mice with Streptomycin Provides a Salmonella enterica Serovar Typhimurium Colitis Model That Allows Analysis of Both Pathogen and Host. Infect. Immun. 71, 2839-2858 (2003).
35. Rivera-Chávez, F. et al. Depletion of Butyrate-Producing Clostridia from the Gut Microbiota Drives an Aerobic Luminal Expansion of Salmonella. Cell Host Microbe 19, 443-454 (2016).
36. Winter, S. E. et al. Gut Inflammation Provides a Respiratory Electron Acceptor for Salmonella. Nature 467, 426-429 (2010).
37. Choi, J. & Groisman, E. A. Acidic pH sensing in the bacterial cytoplasm is required for Salmonella virulence. Mol. Microbiol. (2016) doi:10.1111/mmi.13439.
38. Huang, Y., Suyemoto, M., Garner, C. D., Cicconi, K. M. & Altier, C. Formate acts as a diffusible signal to induce Salmonella invasion. J. Bacteriol. (2008) doi:10.1128/JB.00205-08.
39. Miao, E. A. et al. Cytoplasmic flagellin activates caspase-1 and secretion of interleukin 1β via Ipaf. Nat. Immunol. (2006) doi:10.1038/ni1344.
40. Broz, P. et al. Redundant Roles for Inflammasome Receptors NLRP3 and NLRC4 in Host Defense Against Salmonella. J. Exp. Med. 207, 1745-1755 (2010).
41. Knodler, L. A. et al. Noncanonical Inflammasome Activation of Caspase-4/Caspase-11 Mediates Epithelial Defenses against Enteric Bacterial Pathogens. Cell Host Microbe 16, 249-256 (2014).
42. Sellin, M. E. et al. Epithelium-Intrinsic NAIP/NLRC4 Inflammasome Drives Infected Enterocyte Expulsion to Restrict Salmonella Replication in the Intestinal Mucosa. Cell Host Microbe 16, 237-248 (2014).
43. Chen, L. M., Kaniga, K. & Galán, J. E. Salmonella Spp. Are Cytotoxic for Cultured Macrophages. Mol. Microbiol. 21, 1101-1115 (1996).
44. Monack, D. M., Raupach, B., Hromockyj, A. E. & Falkow, S. Salmonella Typhimurium Invasion Induces Apoptosis in Infected Macrophages. Proc. Natl. Acad. Sci. U.S.A. 93, 9833-9838 (1996).
45. van der Velden, A. W., Lindgren, S. W., Worley, M. J. & Heffron, F. Salmonella Pathogenicity Island 1-Independent Induction of Apoptosis in Infected Macrophages by Salmonella enterica Serotype Typhimurium. Infect. Immun. 68, 5702-5709 (2000).
46. Lee, J.-Y. et al. High-Fat Diet and Antibiotics Cooperatively Impair Mitochondrial Bioenergetics to Trigger Dysbiosis that Exacerbates Pre-inflammatory Bowel Disease. Cell Host Microbe (2020) doi:10.1016/j.chom.2020.06.001.
47. Goretsky, T. et al. P53 mediates TNF-induced epithelial cell apoptosis in IBD. Am. J. Pathol. (2012) doi:10.1016/j.ajpath.2012.06.016.
48. Burton, P. R. et al. The Wellcome Trust Case Control Consortium. Genome-wide association study of 14,000 cases of seven common diseases and 3,000 shared controls. Nature (2007) doi:10.1038/nature05911.
49. Vereecke, L. et al. Enterocyte-specific A20 deficiency sensitizes to tumor necrosis factor-induced toxicity and experimental colitis. J. Exp. Med. (2010) doi:10.1084/jem.20092474.
50. Arnold, J. W. et al. Tumor necrosis factor-α mediates the early pathology in Salmonella infection of the gastrointestinal tract. Microb. Pathog. (1993) doi:10.1006/mpat.1993.1021.
51. Montassier, E. et al. Chemotherapy-driven dysbiosis in the intestinal microbiome. Aliment. Pharmacol. Ther. (2015) doi:10.1111/apt.13302.
52. Lamkanfi, M., Kalai, M., Saelens, X., Declercq, W. & Vandenabeele, P. Caspase-1 activates nuclear factor of the κ-enhancer in B cells independently of its enzymatic activity. J. Biol. Chem. 279, 24785-24793 (2004).
53. Datsenko, K. A. & Wanner, B. L. One-Step Inactivation of Chromosomal Genes in Escherichia Coli K-12 Using PCR Products. Proc. Natl. Acad. Sci. U.S.A. 97, 6640-6645 (2000).
54. Shivak, D. J. et al. A modular, Tn7-based system for making bioluminescent or fluorescent salmonella and Escherichia coli strains. Appl. Environ. Microbiol. (2016) doi:10.1128/AEM.01346-16.
55. Rivera-Chavez, F. et al. Salmonella Uses Energy Taxis to Benefit from Intestinal Inflammation. PLOS Pathog. 9, (2013).
56. Anderson, C. J., Clark, D. E., Adli, M. & Kendall, M. M. Ethanolamine Signaling Promotes Salmonella Niche Recognition and Adaptation During Infection. PLOS Pathog 11, e1005278 (2015).
57. Rowley, C. A., Anderson, C. J. & Kendall, M. M. Ethanolamine influences human commensal Escherichia coli growth, gene expression, and competition with enterohemorrhagic E. Coli O157:H7. MBio (2018) doi:10.1128/mBio.01429-18.
58. Dondelinger, Y. et al. Serine 25 phosphorylation inhibits RIPK1 kinase-dependent cell death in models of infection and inflammation. Nat. Commun. 10, (2019).
59. Poon, I. K. H. et al. Unexpected Link Between an Antibiotic, Pannexin Channels and Apoptosis. Nature 507, 329-334 (2014).
60. Dubois, H. et al. Nlrp3 inflammasome activation and Gasdermin D-driven pyroptosis are immunopathogenic upon gastrointestinal norovirus infection. PLOS Pathog. 15, (2019).
61. Hoiseth, S. K. & Stocker, B. A. Aromatic-Dependent Salmonella Typhimurium Are Non-Virulent and Effective as Live Vaccines. Nature 291, 238-239 (1981).
62. Levine, M. M. et al. Escherichia Coli strains that cause diarrhoea but do not produce heat-labile or heat-stable enterotoxins and are non-invasive. Lancet (1978) doi:10.1016/S0140-6736(78)90299-4.
63. Bakker-Woudenberg, I. A. J. M., van Den Berg, J. C., Vree, T. B., Baars, A. M. & Michel, M. F. Relevance of serum protein binding of cefoxitin and cefazolin to their activities against Klebsiella pneumoniae pneumonia in rats. Antimicrob. Agents Chemother. 28, 654-659 (1985).
64. Ledermann, R., Strebel, S., Kampik, C. & Fischer, H. M. Versatile vectors for efficient mutagenesis of Bradyrhizobium diazoefficiens and other alphaproteobacteria. Appl. Environ. Microbiol. (2016) doi:10.1128/AEM.04085-15.
65. Guzman, L. M., Belin, D., Carson, M. J. & Beckwith, J. Tight regulation, modulation, and high-level expression by vectors containing the arabinose P (BAD) promoter. J. Bacteriol. 177, 4121-4130 (1995).

1. An inhibitor of functional expression of a pyruvate formate lyase, wherein the inhibitor is selected from an antisense oligonucleotide, a gapmer, a shRNA, a siRNA, a CRISPR, a TALEN, or a Zinc-finger nuclease.

2. The inhibitor of functional expression of pyruvate formate lyase of claim 1, wherein the inhibitor is comprised in a medicament.

3. A method of treating a gut disease in subject characterized by excessive apoptosis occurs, the method comprising: administering to the subject the inhibitor of functional expression of pyruvate formate lyase of claim 1.

4. The method according to claim 3, wherein the gut disease is associated with colitis, inflammatory bowel disease, chemotherapy induced mucositis, or bacterial infection.

5. The method according to claim 4, wherein the bacterial infection is an enterobacterial infection.

6. The method according to claim 5, wherein the enterobacterial infection is a food-borne infection.

7. The method according to claim 6, wherein the enterobacterial food-borne infection is is an infection of a Klebsiella species, Escherichia species, or Salmonella species.


SEQUENCE LISTING (45 sequences)

SEQ ID NO: 1 - protein, 760 aa, Salmonella enterica
MSELNEKLATAWEGFTKGDWQNEVNVRDFIQKNYTPYEGDESFLAGATDATTKLWDSVMEGVKQENRTHAPVDFDTSVASTITSHDAGYINKALEKIVGLQTEAPLKRAIIPFGGIKMVEGSCKAYNRELDPMIKKIFTEYRKTHNQGVFDVYTPDILRCRKSGVLTGLPDAYGRGRIIGDYRRVALYGIDYLMKDKFAQFTSLQSDLENGVNLEATIRLREEIAEQHRALGQIKEMAAKYGCDISGPATNAQEAIQWTYFGYLAAVKSQNGAAMSFGRVSTFLDAYIERDLKAGKITEQDAQEMIDHLVMKLRMVRFLRTPEYDELFSGDPIWATESIGGMGVDGRTLVTKNSFRFLNTLYTMGPSPEPNITVLWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNFDEVMDRMDHFMDWLAKQYVTALNVIHYMHDKYSYEASLMALHDRDVIRTMACGIAGLSVAADSLSAIKYAKVKPIRDEDGLAIDFEIEGEYPQFGNNDARVDDMAVDLVERFMKKIQKLTTYRGAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMEHPEKYPQLTIRVSGYAVRFNSLTKEQQQDVITRTFTQTM

SEQ ID NO: 2 - DNA, 70 bp, Artificial Sequence
  Primer
atgaacatgagttctgtcaaaaagatcgggctatttgcctgtactggcgtgtgtaggctggagctgcttc

SEQ ID NO: 3 - DNA, 70 bp, Artificial Sequence
  Primer
gtacttatttcgtattttctttcagcaccttaacggtgtaacgtccgtctcatatgaatatcctccttag

SEQ ID NO: 4 - DNA, 24 bp, Artificial Sequence
  Primer
taggacttatctcaacccattcgt

SEQ ID NO: 5 - DNA, 19 bp, Artificial Sequence
  Primer
ttgatcgcgtggctgtcat

SEQ ID NO: 6 - DNA, 70 bp, Artificial Sequence
  Primer
gtgttacatgtccgagcttaatgaaaagttagccacagcctgggaaggttgtgtaggctggagctgcttc

SEQ ID NO: 7 - DNA, 70 bp, Artificial Sequence
  Primer
cccattacatggtctgcgtgaaggtacgagtaataacgtcctgctgctgtcatatgaatatcctccttag

SEQ ID NO: 8 - DNA, 20 bp, Artificial Sequence
  Primer
ctcacctgaccgtgatgagc

SEQ ID NO: 9 - DNA, 23 bp, Artificial Sequence
  Primer
gtgaaaagtcgagttgtcgcaat

SEQ ID NO: 10 - DNA, 20 bp, Artificial Sequence
  Primer
ccttctatgaaaggttgggc

SEQ ID NO: 11 - DNA, 19 bp, Artificial Sequence
  Primer
tgtccagatagcccagtag

SEQ ID NO: 12 - DNA, 70 bp, Artificial Sequence
  Primer
aatgtagtaccaggcagaaggccgtcgacagacggcctttttttgtgcgcatgtttgatgttatggagca

SEQ ID NO: 13 - DNA, 70 bp, Artificial Sequence
  Primer
ctggatgcgactcaaaagccccgaccgggccagggggatccacctgaataaagccgatctcggcttgaac

SEQ ID NO: 14 - DNA, 20 bp, Artificial Sequence
  Primer
atgtttgatgttatggagca

SEQ ID NO: 15 - DNA, 20 bp, Artificial Sequence
  Primer
aagccgatctcggcttgaac

SEQ ID NO: 16 - DNA, 20 bp, Artificial Sequence
  Primer
cttacattcacgcggaagcc

SEQ ID NO: 17 - DNA, 70 bp, Artificial Sequence
  Primer
cctatagtgctgctttctctacttaacagtgctcgtttacgacctgaattgtgtaggctggagctgcttc

SEQ ID NO: 18 - DNA, 70 bp, Artificial Sequence
  Primer
gcccttatattgtttttataacattcactgacttgctatctgctatctcacatatgaatatcctccttag

SEQ ID NO: 19 - DNA, 20 bp, Artificial Sequence
  Primer
aatggcagaacagcgtcgta

SEQ ID NO: 20 - DNA, 20 bp, Artificial Sequence
  Primer
gcgcccagatccatacatca

SEQ ID NO: 21 - DNA, 70 bp, Artificial Sequence
  Primer
gtcgcaatgcgttcatggttaggtgagggagtcagggcgcaacagtggctgtgtaggctggagctgcttc

SEQ ID NO: 22 - DNA, 70 bp, Artificial Sequence
  Primer
caattcattcttcattgtccgccaactcctcttcgctaaggtcaatacttcatatgaatatcctccttag

SEQ ID NO: 23 - DNA, 22 bp, Artificial Sequence
  Primer
actgctttttaaagcgttggct

SEQ ID NO: 24 - DNA, 20 bp, Artificial Sequence
  Primer
acagcacaactcgcccataa

SEQ ID NO: 25 - DNA, 30 bp, Artificial Sequence
  Primer
ctaggaattcatgtccgagcttaatgaaaa

SEQ ID NO: 26 - DNA, 36 bp, Artificial Sequence
  Primer
ctagaagcttttacatggtctgcgtgaaggtacgag

SEQ ID NO: 27 - DNA, 20 bp, Artificial Sequence
  Primer
aaccatgattggcagggagg

SEQ ID NO: 28 - DNA, 20 bp, Artificial Sequence
  Primer
gttgccagcacttgctcaat

SEQ ID NO: 29 - DNA, 20 bp, Artificial Sequence
  Primer
ctaccaggcgaccgaaattg

SEQ ID NO: 30 - DNA, 21 bp, Artificial Sequence
  Primer
gttcctcagatgcggcataca

SEQ ID NO: 31 - DNA, 20 bp, Artificial Sequence
  Primer
tgaaaaacccgaaacgcacc

SEQ ID NO: 32 - DNA, 20 bp, Artificial Sequence
  Primer
gaacatcccggcgataacct

SEQ ID NO: 33 - DNA, 20 bp, Artificial Sequence
  Primer
atgcgtccggacttcaacaa

SEQ ID NO: 34 - DNA, 20 bp, Artificial Sequence
  Primer
atggttttcgccaggtttgc

SEQ ID NO: 35 - DNA, 20 bp, Artificial Sequence
  Primer
aaaggcgaggatctggaagc

SEQ ID NO: 36 - DNA, 20 bp, Artificial Sequence
  Primer
gccgggcgagagatatcaaa

SEQ ID NO: 37 - DNA, 20 bp, Artificial Sequence
  Primer
ttcgccgctaaagtgtccat

SEQ ID NO: 38 - DNA, 20 bp, Artificial Sequence
  Primer
cgcagcaagcaatagcgtag

SEQ ID NO: 39 - DNA, 20 bp, Artificial Sequence
  Primer
atatgctcggcggtacttgg

SEQ ID NO: 40 - DNA, 20 bp, Artificial Sequence
  Primer
ccgcatcaaaccaatgccag

SEQ ID NO: 41 - DNA, 18 bp, Artificial Sequence
  Primer
cgcggtcgtggttatgtg

SEQ ID NO: 42 - DNA, 20 bp, Artificial Sequence
  Primer
gcgctcatcttcttccgaat

SEQ ID NO: 43 - DNA, 20 bp, Artificial Sequence
  Primer
gcagcaaattcgccagaaga

SEQ ID NO: 44 - DNA, 20 bp, Artificial Sequence
  Primer
tgcaatgacctcttcgctgt

SEQ ID NO: 45 - DNA, 20 bp, Artificial Sequence
  sgRNA
ggagcttgtcagtacatgtg